SEQUENCE LISTING (1 sequence)

SEQ ID NO: 1 - protein, 283 aa, Homo sapiens [sequence omitted]

(19) United States
(12) Patent Application Publication
(10) Pub. No.: US 2025/0276039 A1
(43) Pub. Date: Sep. 4, 2025

(54) METHOD OF ENHANCING ANTIBODY-DEPENDENT CELL-MEDIATED CYTOTOXICITY (ADCC)

(71) Applicant: ALBERT EINSTEIN COLLEGE OF MEDICINE, Bronx, NY (US)

(72) Inventors: Betsy Herold, Bronx, NY (US); Clare Burn, Bronx, NY (US)

(21) Appl. No.: 19/084,026

(22) Filed: Mar. 19, 2025

Related U.S. Application Data
(62) Division of application No. 17/286,073, filed on Apr. 16, 2021, now abandoned.
(60) Provisional application No. 62/900,910, filed on Sep. 16, 2019.
(60) Provisional application No. 62/746,731, filed on Oct. 17, 2018.

(51) Int. Cl. A61K 38/19; C07K 16/28

(52) U.S. Cl. CPC A61K 38/191; C07K 16/2878; C07K 2317/622

(57) ABSTRACT

Methods of preferentially enhancing in a subject an antibody-dependent cell-mediated cytotoxicity (ADCC) antibody response over a neutralizing antibody response to a vaccine for an infectious agent using herpesvirus entry mediator (HVEM) agonists, and related compositions.

### STATEMENT OF GOVERNMENT SUPPORT

This invention was made with government support under grant number AI117321 awarded by the National Institutes of Health. The government has certain rights in the invention.

### BACKGROUND

Over 3.7 billion people worldwide are infected with herpes simplex virus type 1 (HSV-1) and approximately 400 million are infected with HSV type 2 (HSV-2) (Looker et al., 2015; Roberts et al., 2003). HSV-1 is the predominant cause of gingivostomatitis, a leading cause of corneal infectious blindness and fatal sporadic encephalitis, and has emerged as the more common cause of primary genital herpes in the United States and other developed countries (Roberts et al., 2003). HSV-2 remains the leading cause of genital herpes worldwide and is a major cofactor in the HIV epidemic (Looker et al., 2017). Both serotypes establish latency in the dorsal root ganglia (DRG) with relatively frequent subclinical and clinical reactivation resulting in viral shedding and transmission (Corey and Schiffer, 2013).

These epidemiological findings underscore the need for vaccines to prevent primary infection, latency, and recurrence, which could also have a beneficial effect on the HIV epidemic. HSV vaccines that have entered into clinical trials have predominantly been adjuvanted subunit vaccines targeting the major envelope glycoprotein D (gD) alone, or in combination with other viral proteins (Awasthi and Friedman, 2014). These vaccines were designed to elicit neutralizing antibody responses in seronegative participants or boost these responses in HSV seropositive individuals (Stanberry et al., 2002; Belshe et al., 2012; Awasthi and Friedman, 2014). One such vaccine is a recombinant gD vaccine adjuvanted with aluminum (alum) and monophosphoryl lipid A (MPL) (rgD-2/AS04, GlaxoSmithKline). Although the vaccine was protective against HSV-2 disease (but did not prevent establishment of latency) in preclinical studies conducted primarily with laboratory adapted viral strains, clinical trial results were disappointing. In Phase three trials conducted among serodiscordant partners, rgD-2/AS04 protected doubly (HSV-1 and HSV-2) seronegative women, but not men, and failed to protect HSV-1 seropositive men or women (Stanberry et al., 2002). In a subsequent field trial that enrolled only doubly-seronegative women, partial protection against genital HSV-1, but not HSV-2 disease was observed and there was no protection against HSV-1 or HSV-2 infection (Belshe et al., 2012).

A recent different approach has been generation of a single-cycle HSV-2 candidate vaccine deleted in gD (ΔgD-2) (See, e.g., U.S. Pat. No. 9,999,665). This vaccine completely protected female mice from skin and vaginal challenge, and male mice from skin challenge, with high doses of clinical isolates of either serotype and prevented the establishment of latency (Petro et al., 2015; 2016; Burn et al., 2017). Unlike rgD-2/AS04, ΔgD-2 induced high titer antibody responses that were weakly neutralizing, but potently activated Fc gamma-receptors (FcγR) to elicit ADCC and antibody dependent phagocytosis (ADCP). Passive transfer studies confirmed that these antibodies were sufficient to protect naïve mice from lethal HSV challenge (Petro et al., 2015; 2016; Burn et al., 2017). Further, vaccination of female mice with ΔgD-2 vaccine has also been shown to protect their pups from subsequent postnatal challenge through transfer of ADCC antibodies from mother to pup; natural sublethal infection, which elicits a predominantly neutralizing antibody response in female mice (and humans) did not provide significant protection to newborns (Kao et al JID, in press). Moreover, the vaccine is immunogenic in HSV seropositive mice, boosting total and ADCC antibody responses and providing complete protection against subsequent challenge with a clinical isolate of HSV-2 (Burn et al, manuscript in preparation).

The predominance of ADCC responses to ΔgD-2 compared to the neutralizing response to natural infections and other vaccine candidates, suggests that gD may play an immunomodulatory role and skew the immune response away from ADCC, and this was investigated.

### SUMMARY

A method of preferentially enhancing in a subject an antibody-dependent cell-mediated cytotoxicity (ADCC) antibody response to a vaccine for an infectious agent, comprising administering to the subject receiving the vaccine an amount of an herpesvirus entry mediator (HVEM) agonist, a tumor necrosis factor superfamily-14 (TNFSF-14) agonist, or a combination thereof, effective to enhance an ADCC antibody response in the subject.

A method of enhancing antibody-dependent cell-mediated cytotoxicity (ADCC) activity of a vaccine for an infectious agent that elicits a neutralizing antibody response, comprising administering to the subject receiving the vaccine for an infectious agent an amount of an herpesvirus entry mediator (HVEM) agonist, a tumor necrosis factor superfamily-14 (TNFSF-14) agonist, or a combination thereof, effective to enhance an ADCC activity in the subject.

A composition comprising a vaccine for an infectious agent and an amount of an herpesvirus entry mediator (HVEM) agonist effective to enhance an ADCC antibody response over a neutralizing antibody response.

A kit for enhancing vaccine response comprising:
(i) an amount of an HVEM agonist; and
(ii) an amount of a vaccine for an infectious agent.

An amount of a herpesvirus entry mediator (HVEM) agonist effective to preferentially enhance in a subject an antibody-dependent cell-mediated cytotoxicity (ADCC) antibody response over a neutralizing antibody response to a vaccine for an infectious agent.

A method of decreasing or blocking Fc-gamma receptor (FcγR)-mediated killing of self-antigen in a subject having an autoimmune disease comprising administering to the subject an amount of an HVEM antagonist, a soluble herpes simplex virus (HSV) glycoprotein D, an antibody binding HVEM, or a combination thereof, effective to decrease or block the FcγR-mediated killing in the subject.

A method of blocking Fc-gamma receptor (FcγR)-mediated killing of self-antigen in a subject having an autoimmune disease comprising administering to the subject an amount of an HVEM antagonist, a soluble herpes simplex virus (HSV) glycoprotein D, an antibody binding HVEM, or a combination thereof, effective to reduce the autoimmune disease in the subject.

### BRIEF DESCRIPTION OF THE DRAWINGS

FIGS. 1A to 1D: ΔgD-2 and rgD-2 differentially protect from infection with clinical isolates of HSV. C57BL/6 mice were vaccinated with 5×10⁶, 5×10⁵ or 5×10⁴ pfu ΔgD-2 or with 5 μg rgD-2-AS04 (provided by GSK) or rgD-2 with alum and MPL (rgD-2+alum/MPL). FIG. 1A and FIG. 1B are graphs of percent survival versus time (days), showing the survival of mice following challenge on the skin with a 10× lethal dose (LD90) of HSV-1 strain B³x1.1 (open symbols, FIG. 1A) or HSV-2 strain SD90 (closed symbols, FIG. 1B). For FIG. 1A, ΔgD-2 5×10⁶, ΔgD-2 5×10⁵ or ΔgD-2 5×10⁴ pfu are significant compared to control. In FIG. 1B, all vaccinations offer significant survival benefit compared to VD60 alone. Survival curves analyzed by Gehan-Breslow-Wilcoxon test; Bonferroni correction used to adjust for multiple testing. FIG. 1C is a graph of copies of HSV-1 or HSV-2 DNA (per gram of tissue) versus challenge strain (HSV-1 B3¹x1 or HSV-2 strain SD90), showing amount of HSV DNA detected in the dorsal root ganglia (DRG) of mice at time of sacrifice (control, rgD-2) or at day 14 post-challenge (ΔgD, rgD-2). Data is displayed as copies per gram of tissue. **p<0.01, ****p<0.0001 by two-way ANOVA. FIG. 1D is a graph showing the percentage survival following passive transfer of 750 μg total IgG from ΔgD-2, rgD-2 or VD60 vaccinated mice to naïve mice 1 day before challenge on the skin with an LD90 of HSV-2 clinical strain 4674. **p-0.0010 compared to VD60, ΔgD-2 also significant compared to rgD-2 (0.0031) by Gehan-Breslow-Wilcoxon test; Bonferroni correction used to adjust for multiple testing. n=10/group; two independent experiments, except for rgD-2 n=5 and passive transfer n=5/group.

FIGS. 2A to 2F: ΔgD-2 and rgD-2 induce functionally different antibody responses to HSV. WT (C57B16) mice were vaccinated with 5×10⁶, 5×10⁵ or 5×10⁴ pfu ΔgD-2or 5 μg rgD-2. One week after boost vaccination, serum samples were collected and HSV-2 specific IgG titer (1:90 000 dilution; FIG. 2A) or total gD-2 binding IgG (FIG. 2B) were quantified by ELISA. FIG. 2C is a graph of neutralization titer to HSV-1 and HSV-2 (C). FIG. 2D is a graph showing the increase (fold induction) in FcγRIV activation as measured using a Promega FcR reporter assay, with cells infected with HSV-1 or HSV-2 as target cells and effector cells expressing FcγRIV and a luciferase reporter. FIG. 2E shows total HSV-2 binding IgG isotypes as assessed by ELISA (1:1000 dilution). FIG. 2F is a graph showing the percentage survival following passive transfer of 750 μg total immune serum from ΔgD-2-vaccinated mice to naïve WT or FcγRIV−/− (FcγRIV KO) mice. *p<0.05; **p<0.01, ****p<0.0001; n=10/group; two independent experiments, except rgD-2 and FcγRIV−/−n=5/group. Statistical analysis by ANOVA except survival curve analyzed by Gehan-Breslow-Wilcoxon test. In FIG. 2F ΔgD-2 WT is significantly different from VD60 (p=0.0010) and ΔgD-2 FcγRIV KO (p=0.0039).

FIGS. 3A to 3E: Vaccination of HVEM−/− mice with ΔgD-2 or rgD-2 abrogates protection. WT or HVEM−/− mice were prime-boost vaccinated with 5×10⁵ pfu ΔgD-2 or 5 μg rgD-2 with alum and MPL at a three week interval. FIG. 3A shows percentage survival following skin challenge with 10×LD₉₀ HSV-2 SD90. Survival curves analyzed by Gehan-Breslow-Wilcoxon test, ΔgD-2 WT significantly different from ΔgD-2 KO, rgD-2 and VD60 (P<0.0001) Serum was collected 7 days after boost vaccination and antibody responses were assessed. FIG. 3B shows total HSV-2-binding ELISA serum titer; FIG. 3C shows HSV-2 neutralization antibody titer, FIG. 3D shows fold induction of FcγRIV activation, and FIG. 3E shows the IgG isotypes, each of which was measured as assessed by ELISA (1:1000 dilution). *p<0.05, **p<0.01, ***p<0.001, ****p<0.0001; n=10-20 animals per group. Data is from two independent experiments

FIG. 4A to 4B: HVEM is involved in mounting and mediating ADCC. FIG. 4A shows the percentage survival of WT mice following passive transfer of 750 μg total immune serum from WT or HVEM−/− mice prime/boost immunized with VD60 (control) or ΔgD-2. FIG. 4B shows the percent survival of HVEM−/− mice following passive transfer of 750 μg total immune serum from WT mice immunized with VD60 (control) or ΔgD-2.

FIGS. 5A to 5D: Evaluation in BTLA−/− mice. WT (C57B1/6) mice or BTLA−/− mice were prime-boost vaccinated (3 week interval) with 5×10⁵ pfu ΔgD-2, VD-60 cell lysate (control) or 5 μg rgD-2 with alum and MPL. One week following boost vaccination, serum was obtained by retroorbital bleed and mice were challenged with 10×LD₉₀ of strain SD90. FIG. 5A shows total HSV-2 specific IgG, FIG. 5B shows total HSV-2 neutralization antibody titer, FIG. 5C shows fold induction of FcγRIV activation, and FIG. 5D shows percentage survival following skin challenge with 10×LD₉₀ SD90.

FIGS. 6A to 6D: Evaluation in LIGHT−/− mice. WT (C57B1/6) mice or LIGHT−/− mice were prime-boost vaccinated (3 week interval) with 5×10⁵ pfu ΔgD-2 or 5 VD-60 cell lysate (control). One week following boost vaccination, serum was obtained by retroorbital bleed and mice were challenged with 10×LD₉₀ of HSV-2 strain SD90. FIG. 6A shows total HSV-2 specific IgG, FIG. 6B shows total HSV-2 neutralization antibody titer, FIG. 6C shows fold induction of FcγRIV activation, and FIG. 6D shows percentage survival following skin challenge with 10×LD₉₀ SD90.

FIG. 7A to 7B: Cells derived from HVEM−/− mice are deficient in effector function. Total bone marrow cells (FIG. 7A) or bone marrow-derived cells stimulated with GM-CSF for 7 days (FIG. 7B) were used in an ex vivo ADCC assay. Data is shown as percentage change in dead cells compared to a “no serum” control. Each point indicates a single mouse serum or antibody sample. *p<0.05, **p<0.01, ***p<0.001, ****p<0.0001 by Mann-Whitney Test.

FIGS. 8A to 8C: gD (soluble protein or on viral envelope) and anti-HVEM antibodies inhibit FcγRIV activation. In FIG. 8A, HSV-2 infected cells were the target; immune serum from ΔgD-2 or VD-60 (control) immunized mice were added in the absence or presence of recombinant gD protein, recombinant gB protein, or a commercial anti-HVEM Ab and then incubated with effector cells (PROMEGA, mFcγRIV-expressing linked to NFAT luciferase reporter). Soluble gD or anti-HVEM reduces FcR activation. In FIG. 8B, the target cells were infected with WT HSV-2 SD90 or ΔgD-2 (so the target does not have gD expressed on the infected cell) and incubated with effector cells. When there is no gD in the target cell, FcR activation is enhanced; adding recombinant gD protein brings FcR activation back down. In FIG. 8C, the assay was performed using Raji cells (express CD20) and rituximab (anti-CD20) in the absence or presence of anti-HVEM antibody (10 or 20 μg). *p<0.05, **p<0.01, ***p<0.001, by paired T test (A) and ANOVA (B).

FIG. 9: gD protein and anti-HVEM antibody inhibit hFcγRIIIa activation in response to human immune serum. Immune serum from HSV seropositive individuals was combined with ΔgD-2 infected target cells and effector cells expressing hFcγRIIIa, alone (human HSV+serum) or in combination with 5 μg or 10 μg of gD or anti-HVEM antibody. The serum has low levels (5-6-fold) of FcR activating Abs (human kit) and killing is decreased when recombinant gD protein is added.

FIGS. 10A to 10B. HVEM is needed for generating ADCC Abs against other pathogens. Wild-type or HVEM−/− knockout mice were immunized with VSV-Ebola virus construct (Chandran lab) and the immune serum assayed for total and ADCC activating Abs. FIG. 10A is a graph showing total antibody to the Ebola protein and FIG. 10B is a graph of FcγRIV activation. Again, there is a decrease in FcR activating Abs in HVEM KO mice. (*VSV-Ebola vaccine gift from K. Chandran.)

FIGS. 11A to 11B: Passive transfer suggests a role for LIGHT not BTLA. WT (C57B16), BTLA−/−, or LIGHT−/− mice received an intraperitoneal administration of 750 μg serum isolated from ΔgD-2 or VD60 immunized WT mice one day prior to challenge with 10×LD₉₀ SD90. FIG. 11A shows the results in WT and BTLA−/− mice, and FIG. 11B shows the results in WT and LIGHT−/− mice.

### DETAILED DESCRIPTION

A method of preferentially enhancing in a subject an antibody-dependent cell-mediated cytotoxicity (ADCC) antibody response to a vaccine for an infectious agent, comprising administering to the subject receiving the vaccine an amount of an herpesvirus entry mediator (HVEM) agonist, a tumor necrosis factor superfamily-14 (TNFSF-14) agonist, or a combination thereof, effective to enhance an ADCC antibody response in the subject.

A method of preferentially enhancing in a subject an antibody-dependent cell-mediated cytotoxicity (ADCC) antibody response to a vaccine for an infectious agent, comprising administering to the subject receiving the vaccine an amount of an herpesvirus entry mediator (HVEM) agonist effective to enhance an ADCC antibody response in the subject.

A method of preferentially enhancing in a subject an antibody-dependent cell-mediated cytotoxicity (ADCC) antibody response over a neutralizing antibody response to a vaccine for an infectious agent, comprising administering to the subject receiving the vaccine an amount of an HVEM agonist, a tumor necrosis factor superfamily-14 (TNFSF-14) agonist, or a combination thereof, effective to enhance an ADCC antibody response over a neutralizing antibody response.

A method of enhancing antibody-dependent cell-mediated cytotoxicity (ADCC) activity of a vaccine for an infectious agent that elicits a neutralizing antibody response, comprising administering to the subject receiving the vaccine for an infectious agent an amount of an herpesvirus entry mediator (HVEM) agonist, a tumor necrosis factor superfamily-14(TNFSF-14) agonist, or a combination thereof, effective to enhance an ADCC activity in the subject.

A method of enhancing antibody-dependent cell-mediated cytotoxicity (ADCC) activity of a vaccine for an infectious agent that elicits a neutralizing antibody response, comprising administering to the subject receiving the vaccine for an infectious agent an amount of an herpesvirus entry mediator (HVEM) agonist effective to enhance an ADCC activity in the subject

As used herein “preferentially enhancing” or “enhancing” in a subject an antibody-dependent cell-mediated cytotoxicity (ADCC) antibody response means promoting or increasing or amplifying an ADCC response over its resting level in the subject (prior to HVEM and/or TNFSF-14 agonist administration). The amount or value to which the ADCC antibody response is enhanced is greater than the amount or value to which the neutralizing antibody response in the same subject is enhanced, if at all, over its resting level in the subject (prior to HVEM and/or TNFSF-14 agonist administration). For example, the term “preferentially enhancing” refers to an increase in an ADCC antibody response in a subject.

In embodiments, to promote the generation of the ADCC response, the HVEM and/or TNFSF-14 agonist is administered to the subject at substantially the same time as the vaccine. For example, the HVEM and/or TNFSF-14 agonist is combined with the vaccine prior to administration and is administered simultaneously with the vaccine. Alternatively, the HVEM and/or TNFSF-14 agonist is administered to the subject before or after administration of the vaccine to the subject such that the period of time between administration of the vaccine and administration of the HVEM and/or TNFSF-14 agonist does not affect the ability of the HVEM agonist to enhance ADCC activity in a subject or to enhance an ADCC antibody response.

In embodiments, the agonist (HVEM and/or TNFSF-14 agonist) can be administered to a subject infected with an infectious agent, alone or in combination with Fc-gamma receptor (FcγR) activating antibody, in order to enhance an ADCC response in the subject. The FcγR activating antibody can be present in immune serum or can be an isolated antibody. For example, the isolated antibody can be a monoclonal antibody such as rituximab which acts via FcγR in the presence of the HVEM and/or TNFSF-14 agonist.

The tumor necrosis factor superfamily-14 (TNFSF-14) protein, also known as LIGHT (homologous to Lymphotoxin, exhibits Inducible expression and competes with HSV Glycoprotein D for binding to Herpesvirus entry mediator, a receptor expressed on T lymphocytes), is a secreted protein of the TNF superfamily and a ligand for HVEM. The terms TNFSF-14 and LIGHT can be used interchangeably.

In embodiments, the HVEM agonist comprises a TNFSF-14 protein or a portion thereof. For example, the TNFSF-14 protein can be a full length protein or a portion of the TNFSF-14 protein which binds to HVEM.

In embodiments, a method of preferentially enhancing in a subject an antibody-dependent cell-mediated cytotoxicity (ADCC) antibody response to a vaccine for an infectious agent, comprises administering to the subject receiving the vaccine an amount TNFSR-14 protein or a portion thereof effective to enhance an ADCC antibody response.

In embodiments of the methods, the HVEM agonist is a TNFSF hexavalent fusion protein. Hexavalent TNFSF fusion proteins are known in the art. (See, for example, Gieffers et al., Proceedings of the AACR Special Conference on Tumor Immunology and Immunotherapy; 2016 Oct 20-23; Cancer Immunol Res 2017;5(3 Suppl):Abstract nr. A83, hereby incorporated by reference. See for example, apogenix.com/en/immuno-oncology/hera-technology-platform, hereby incorporated by reference.)

In embodiments, the HVEM agonist is a TNFSF hexavalent fusion protein comprises a single chain polypeptide comprising three LIGHT (TNFSF-14) subsequences folded into a functional trivalent receptor binding domain, fused at a C-terminus thereof to a silenced IgG1 Fc-domain as a dimerization scaffold.

In embodiments, the HVEM agonist comprises an agonist antibody which binds, and activates, HVEM.

In embodiments, the HVEM agonist is a single-chain variable fragment (scFv) of an agonist monoclonal antibody which binds, and activates, HVEM.

In embodiments, the subject is not administered an additional immunostimulatory agent.

In embodiments, the subject is not administered an additional tumor necrosis factor superfamily (TNFSF) superfamily stimulatory agent or TNFSF inhibitory agent.

In embodiments, the subject is not administered a toll-like receptor (TLR) agonist (e.g., a TLR3 agonist, a TLR4 agonist, a TLR8 agonist, a TLR9 agonist, or a TLR9 agonist), a CD40 agonist, a CD27 agonist, a MDA5 agonist, a nucleotide-binding oligomerization domain-containing protein agonist (e.g., NOD1, NOD2 agonist), or a combinations thereof.

In embodiments, the subject is not administered a TLR agonist, a domain present in neuronal apoptosis inhibitory protein (NAIP), a domain present in class II transactivator (CIITA), a domain present in hydroxyeicosatetraenoic acid (HET-E), or a domain present in TP-1(NACHT)-Leucine Rich Repeat (LRR), a domain present in a nod-like receptor (NLR) agonist, a retinoic acid-inducible gene-I RIG-like helicase (RLH) agonist, a cytokine/chemokine receptor agonist, a purinergic receptor agonist, or a combinations thereof.

In embodiments, the vaccine is not a DNA vaccine or a genetic vaccine.

In embodiments, the vaccine is not a cancer vaccine, an anti-tumor vaccine, or a vaccine for a target on a tumor.

In embodiments, the vaccine is a vaccine against an infectious agent (infectious pathogen).

In embodiments, the infectious agent is a virus, a bacteria, fungus, or parasite or a combination thereof.

In embodiments, the method elicits and/or increases (enhances) production of Fc gamma receptor IV (FcγRIV)-binding antibodies.

A composition is provided comprising a vaccine for an infectious agent and an amount of an herpesvirus entry mediator (HVEM) agonist effective to enhance an ADCC antibody response over a neutralizing antibody response.

In embodiments, the HVEM agonist is a hexavalent TNFSF superfamily fusion protein.

In embodiments, the TNFSF superfamily hexavalent fusion protein comprises a single chain polypeptide comprising three TNFSF-14 (LIGHT) subsequences folded into a functional trivalent receptor binding domain, fused at a C-terminus thereof to a silenced IgG1 Fc-domain as a dimerization scaffold.

In embodiments, the HVEM agonist is an agonist antibody which binds IIVEM.

In embodiments, the HVEM agonist is a single-chain variable fragment (scFv) of an agonistic monoclonal antibody which binds HVEM.

In embodiments, the infectious agent is at least one of rubeola virus, rubella virus, Vibrio cholera, Neisseria meningitidis, influenza virus, Corynebacterium diptheriae, rubulavirus, cytomegalovirus, Clostridium tetani, hepatitis A virus, hepatitis b virus, hepatitis E virus, Bordatella pertussis, Mycobacterium tuberculosis, Streptococcus pneumoniae, Salmonella enterica serotype Typhi, poliovirus, tick-born encephalitis virus (TBEV), Haemophilis influenza type b, rabies virus, varicella-zoster virus, human papilloma virus, rotavirus, flaviviridae, Plasmodium falciparum, dengue virus, alphavirus, HSV-1, HSV-2, ebola virus, vesicular stomatitis virus (VSV), Bacillus anthracis, Yersinia pestis, Coxiella burnetti, variola virus, or a combination thereof. However, the infectious agent which may be the target of a vaccine for the infectious agent, are not limited thereto.

In embodiments, the vaccine is for at least one of Measles, Rubella, Cholera, Meningococcal disease, Influenza, Diphtheria, Mumps, Tetanus, Hepatitis A, Pertussis, Tuberculosis, Hepatitis B, Pneumoccocal disease, Typhoid fever, Hepatitis E, Poliomyelitis, Tick-borne encephalitis, Haemophilus influenzae type b, Rabies, Varicella and herpes zoster (shingles), Human papilloma-virus, Rotavirus gastroenteritis, Yellow fever, Japanese encephalitis, Malaria, Dengue fever, Anthrax, Plague, Q fever, Smallpox, HSV-1, VSV, ebola hemorrhagic fever, Eastern Equine Encephalitis, Western Equine Encephalitis, Venezuelan Equine Encephalitis, or HSV-2. However, vaccine or the disease(s) targeted by the vaccine are not limited thereto.

In embodiments, the isolated antibody or antigen-binding fragment thereof is chimeric or humanized.

In embodiments, the isolated antibody or antigen-binding fragment thereof comprises a monoclonal antibody, an scFv, an Fab fragment, an Fab′ fragment, an F(ab)′ fragment, or a combination thereof. It is noted that while a scFv is not strictly a fragment of an antibody, rather it is a fusion protein, herein a fragment of an antibody includes an scFv unless otherwise excluded.

In embodiments, the subject does not have cancer. In embodiments, the subject does not have a hematological cancer. In embodiments, the subject does not have a solid tumor. In embodiments, the subject has not been diagnosed with a cancer.

In an embodiment, the composition is a pharmaceutical or biologic composition and comprises a carrier which is a pharmaceutical carrier. A pharmaceutical composition comprising the antibody or binding fragment thereof, described herein, and a pharmaceutically acceptable excipient, is also provided.

A kit for enhancing vaccine response comprising:
(i) an amount of an HVEM agonist; and
(ii) an amount of a vaccine for an infectious agent.

In embodiments, the infectious agent is at least one of rubeola virus, rubella virus, Vibrio cholera, Neisseria meningitidis, influenza virus, Corynebacterium diptheriae, rubulavirus, cytomegalovirus, Clostridium tetani, hepatitis A virus, hepatitis b virus, hepatitis E virus, Bordatella pertussis, Mycobacterium tuberculosis, Streptococcus pneumoniae, Salmonella enterica serotype Typhi, poliovirus, tick-born encephalitis virus (TBEV), Haemophilis influenza type b, rabies virus, varicella-zoster virus, human papilloma virus, rotavirus, flaviviridae, Plasmodium falciparum, dengue virus, alphavirus, HSV-1, HSV-2, ebola virus, vesicular stomatitis virus (VSV), Bacillus anthracis, Yersinia pestis, Coxiella burnetti, variola virus, or a combination thereof. However, the infectious agent which may be the target of a vaccine for the infectious agent, are not limited thereto.

In embodiments, the vaccine is for at least one of Measles, Rubella, Cholera, Meningococcal disease, Influenza, Diphtheria, Mumps, Tetanus, Hepatitis A, Pertussis, Tuberculosis, Hepatitis B, Pneumoccocal disease, Typhoid fever, Hepatitis E, Poliomyelitis, Tick-borne encephalitis, Haemophilus influenzae type b, Rabies, Varicella and herpes zoster (shingles), Human papilloma-virus, Rotavirus gastroenteritis, Yellow fever, Japanese encephalitis, Malaria, Dengue fever, Anthrax, Plague, Q fever, Smallpox, HSV-1, VSV, ebola hemorrhagic fever, Eastern Equine Encephalitis, Western Equine Encephalitis, Venezuelan Equine Encephalitis, or HSV-2. However, vaccine or the disease(s) targeted by the vaccine are not limited thereto.

In embodiments, the vaccine comprises a herpes simplex virus-2 (HSV-2) having a deletion of the HSV-2 glycoprotein D-encoding gene in the genome thereof, wherein the deletion of the HSV-2 glycoprotein D-encoding gene in the genome thereof comprises a partial deletion of the HSV-2 glycoprotein D-encoding gene or a deletion of the entire HSV-2 glycoprotein D-encoding (gD) gene and wherein the recombinant HSV-2 is phenotypically complemented with a herpes simplex virus-1 (HSV-1) glycoprotein D by propagating the recombinant HSV-2 in a complementing cell expressing the HSV-1 glycoprotein D.

In embodiments, the HSV-2 having the deletion of the HSV-2 gD-encoding gene in the genome thereof is a recombinant HSV-2.

The HSV-2 having a deletion of the HSV-2 glycoprotein D-encoding gene in the genome thereof is a single-cycle virus. A partial deletion of the gD-encoding gene in the HSV-2 having a deletion of the HSV-2 glycoprotein D-encoding gene in the genome thereof, is such that if gD is expressed/produced, the protein is unable to bind to and activate HVEM, and is unable to facilitate entry of the HSV-2 into a host cell.

In embodiments, the HVEM agonist is a hexavalent TNFSF fusion protein.

In embodiments, the HVEM agonist is a TNFSF hexavalent fusion protein comprises a single chain polypeptide comprising three TNFSF-14 (LIGHT) subsequences folded into a functional trivalent receptor binding domain, fused at a C-terminus thereof to a silenced IgG1 Fc-domain as a dimerization scaffold.

In embodiments, the HVEM agonist is an agonist antibody which binds HVEM.

In embodiments, the HVEM agonist is a single-chain variable fragment (scFv) of anti-HVEM agonistic monoclonal antibody.

An amount of a herpesvirus entry mediator (HVEM) agonist effective to preferentially enhance in a subject an antibody-dependent cell-mediated cytotoxicity (ADCC) antibody response over a neutralizing antibody response to a vaccine for an infectious agent.

An amount of a herpesvirus entry mediator (HVEM) agonist for preferentially enhancing in a subject, in response to a vaccine, an antibody-dependent cell-mediated cytotoxicity (ADCC) antibody response over a neutralizing antibody response to a vaccine for an infectious agent.

Use of an amount of a herpesvirus entry mediator (HVEM) agonist for the manufacture of a medicament to preferentially enhance in a subject an antibody-dependent cell-mediated cytotoxicity (ADCC) antibody response.

Use of an amount of a herpesvirus entry mediator (HVEM) agonist for the manufacture of a medicament to preferentially enhance in a subject, in response to a vaccine, an antibody-dependent cell-mediated cytotoxicity (ADCC) antibody response.

In embodiments, a method of decreasing or blocking Fc-gamma receptor (FcγR)-mediated killing (or ADCC) of self-antigen in a subject having an autoimmune disease comprises administering to the subject an amount of an HVEM antagonist, a soluble herpes simplex virus (HSV) glycoprotein D, an antibody binding HVEM, or a combination thereof, effective to decrease or block the FcγR-mediated killing in the subject.

In embodiments, a method of decreasing or blocking Fc-gamma receptor (FcγR)-mediated killing (or ADCC) of self-antigen in a subject having an autoimmune disease comprises administering to the subject an amount of an HVEM antagonist, a soluble herpes simplex virus (HSV) glycoprotein D, an antibody binding HVEM, or a combination thereof, effective to reduce the autoimmune disease in the subject.

In embodiments, a method of blocking Fc-gamma receptor (FcγR)-mediated killing (or ADCC) of self-antigen in a subject having an autoimmune disease comprises administering to the subject an amount of an HVEM antagonist, a soluble herpes simplex virus (HSV) glycoprotein D, an antibody binding HVEM, or a combination thereof, effective to reduce the autoimmune disease in the subject.

In embodiments, the Fc-gamma receptor (FcγR)-mediated killing of self-antigen refers to the FcγR-mediated killing (ADCC) of cells expressing self-antigen.

As used herein decreasing FcγR-mediated killing or ADCC in a subject means decreasing to any extent the amount or value of FcγR-mediated killing or ADCC of cells expressing self-antigen over its resting level in the subject (prior to antagonist administration), while blocking FcγR-mediated killing or ADCC in a subject means substantially preventing the FcγR-mediated killing or ADCC of cells expressing self-antigen in the subject.

In embodiments, the HSV glycoprotein D is HSV-1 glycoprotein D, HSV-2 glycoprotein D, or a combination thereof. The glycoprotein D can be used alone, linked to a carrier, or as part of a fusion protein. The glycoprotein D can be the full length protein or a portion of the glycoprotein D protein which binds to the HVEM receptor. For example, the HSV glycoprotein is soluble, full length HSV-1 glycoprotein D, HSV-2 glycoprotein D, or a combination thereof. The HSV glycoprotein D or the portion which binds to the HVEM receptor, can be a recombinant protein.

In embodiments, the antibody binding HVEM (anti-HVEM antibody) is an antibody which binds to, and does not activate HVEM, and which prevents or blocks attachment of a ligand to HVEM.

In embodiments, the autoimmune disease comprises, for example, rheumatoid arthritis, multiple sclerosis, type 1 diabetes mellitus, autoimmune hepatitis, Sjorgren's syndrome, systemic lupus erythematosus, inflammatory bowel disease, Guillain-Barre syndrome, psoriasis, Grave's disease, Hashimoto's thyroiditis, myasthenia gravis, vasculitis, or a combination thereof, but is not limited thereto, and any autoimmune disease which benefits from a decrease in ADCC may be treated with the soluble HSV glycoprotein D, the HVEM antagonist, or the antibody binding HVEM.

As used herein, the term “antibody” refers to an intact antibody, i.e. with complete Fc and Fv regions. “Fragment” refers to any portion of an antibody, or portions of an antibody linked together, such as, in non-limiting examples, a Fab, F(ab)2, a single-chain Fv (scFv), which is less than the whole antibody, but which comprises an antigen-binding portion and which competes with the intact antibody of which it is a fragment for specific binding to the antigen. In this case, the antigen is HVEM, preferably human HVEM.

Such fragments can be prepared, for example, by cleaving an intact antibody or by recombinant means. (See generally, Fundamental Immunology, Ch. 7, Paul, W., ed., 2nd ed. Raven Press, N.Y. (1989), hereby incorporated by reference in its entirety). Antigen-binding fragments may be produced by recombinant DNA techniques or by enzymatic or chemical cleavage of intact antibodies or by molecular biology techniques. In some embodiments, a fragment is an Fab, Fab′, F(ab′)2, Fd, Fv, complementarity determining region (CDR) fragment, single-chain antibody (scFv), (a variable domain light chain (VL) and a variable domain heavy chain (VH) linked via a peptide linker. In an embodiment, the scFv comprises a variable domain framework sequence having a sequence identical to a human variable domain FR1, FR2, FR3 or FR4. In an embodiment, the scFv comprises a linker peptide from 5 to 30 amino acid residues long. In an embodiment, the scFv comprises a linker peptide comprising one or more of glycine, serine and threonine residues.

In an embodiment the linker of the scFv is 10-25 amino acids in length. In an embodiment the peptide linker comprises glycine, serine and/or threonine residues. For example, see Bird et al., Science, 242:423-426 (1988) and Huston et al., Proc. Natl. Acad. Sci. USA, 85:5879-5883 (1988) each of which are hereby incorporated by reference in their entirety), or a polypeptide that contains at least a portion of an antibody that is sufficient to confer human HVEM specific antigen binding on the polypeptide, including a diabody. From N-terminus to C-terminus, both the mature light and heavy chain variable domains comprise the regions FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987), or Chothia et al., Nature 342:878-883 (1989), each of which are hereby incorporated by reference in their entirety). As used herein, the term “polypeptide” encompasses native or artificial proteins, protein fragments and polypeptide analogs of a protein sequence. A polypeptide may be monomeric or polymeric. As used herein, an Fd fragment means an antibody fragment that consists of the VH and CHI domains; an Fv fragment consists of the V1 and VH domains of a single arm of an antibody; and a dAb fragment (Ward et al., Nature 341:544-546 (1989) hereby incorporated by reference in its entirety) consists of a VH domain. In some embodiments, fragments are at least 5, 6, 8 or 10 amino acids long. In other embodiments, the fragments are at least 14, at least 20, at least 50, or at least 70, 80, 90, 100, 150 or 200 amino acids long.

The term “monoclonal antibody” as used herein refers to an antibody member of a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a human HVEM. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. Thus an identified monoclonal antibody can be produced by non-hybridoma techniques, e.g. by appropriate recombinant means once the sequence thereof is identified.

In an embodiment of the inventions described herein, the antibody is isolated. As used herein, the term “isolated antibody” refers to an antibody that by virtue of its origin or source of derivation has one, two, three or four of the following: (1) is not associated with naturally associated components that accompany it in its native state, (2) is free of other proteins from the same species, (3) is expressed by a cell from a different species, and (4) does not occur in nature absent the hand of man.

In an embodiment the antibody is humanized. “Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region (HVR) (or CDR) of the recipient are replaced by residues from a HVR (or CDR) of a non-human species (donor antibody) such as mouse, rat, rabbit, or nonhuman primate having the desired specificity, affinity, and/or capacity. In a preferred embodiment, framework (FR) residues of the murine mAb are replaced with corresponding human immunoglobulin variable domain framework (FR) residues. These may be modified further in embodiments to further refine antibody performance. Furthermore, in a specific embodiment, humanized antibodies may comprise residues that are not found in the recipient antibody or in the donor antibody. In an embodiment, the humanized antibodies do not comprise residues that are not found in the recipient antibody or in the donor antibody. In general, a humanized antibody will comprise substantially all of at least one, and typically two, variable domains, in which all, or in embodiments substantially all, of the hypervariable loops correspond to those of a non-human immunoglobulin, and all, or in embodiments substantially all, of the FRs are those of a human immunoglobulin sequence. The humanized antibody optionally will also comprise at least a portion of an immunoglobulin constant region (Fc), typically that of a human immunoglobulin. See, e.g., Jones et al., Nature 321:522-525 (1986); Riechmann et al., Nature 332:323-329 (1988); Presta, Curr. Op. Struct. Biol. 2:593-596 (1992); Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409, the contents of each of which references and patents are hereby incorporated by reference in their entirety. In one embodiment where the humanized antibodies do comprise residues that are not found in the recipient antibody or in the donor antibody, the Fc regions of the antibodies are modified as described in WO 99/58572, the content of which is hereby incorporated by reference in its entirety.

Techniques to humanize a monoclonal antibody are well known and are described in, for example, U.S. Pat. Nos. 4,816,567; 5,807,715; 5,866,692; 6,331,415; 5,530,101; 5,693,761; 5,693,762; 5,585,089; and 6,180,370, the content of each of which is hereby incorporated by reference in its entirety. A number of “humanized” antibody molecules comprising an antigen-binding site derived from a non-human immunoglobulin have been described, including antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J. Immunol. 138:4534-4538 (1987), and Brown et al. Cancer Res. 47:3577-3583 (1987), the content of each of which is hereby incorporated by reference in its entirety. Other references describe rodent hypervariable regions or CDRs grafted into a human supporting framework region (FR) prior to fusion with an appropriate human antibody constant domain. See, for example, Riechmann et al. Nature 332:323-327 (1988), Verhoeyen et al. Science 239:1534-1536 (1988), and Jones et al. Nature 321:522-525 (1986), the content of each of which is hereby incorporated by reference in its entirety. Another reference describes rodent CDRs supported by recombinantly veneered rodent framework regions-European Patent Publication No. 0519596 (incorporated by reference in its entirety). These “humanized” molecules are designed to minimize unwanted immunological response toward rodent anti-human antibody molecules which limits the duration and effectiveness of therapeutic applications of those moieties in human recipients. The antibody constant region can be engineered such that it is immunologically inert (e.g., does not trigger complement lysis). See, e.g. PCT Publication No. WO99/58572; UK Patent Application No. 9809951.8. Other methods of humanizing antibodies that may also be utilized are disclosed by Daugherty et al., Nucl. Acids Res. 19:2471-2476 (1991) and in U.S. Pat. Nos. 6,180,377; 6,054,297; 5,997,867; 5,866,692; 6,210,671; and 6,350,861; and in PCT Publication No. WO 01/27160 (each incorporated by reference in their entirety).

In embodiments, the antibodies or fragments herein can be produced recombinantly, for example antibodies expressed using a recombinant expression vector transfected into a host cell, antibodies isolated from a recombinant, combinatorial human antibody library, antibodies isolated from an animal (e.g., a mouse) that is transgenic for human immunoglobulin genes.

In an embodiment, the anti-HVEM antibody described herein is capable of specifically binding (or specifically binds) a human HVEM and eliciting an agonistic response. As used herein, the terms “is capable of specifically binding” or “specifically binds” refers to the property of an antibody or fragment of binding to the (specified) antigen with a dissociation constant that is <1 μM, preferably <1 nM and most preferably <10 pM. In an embodiment, the Kd of the antibody (or fragment) for HVEM is better than 10.0 nM. In an embodiment, the Kd of the antibody (or fragment) for HVEM is better than 1.0 nM. In an embodiment, the Kd of the antibody (or fragment) for HVEM is better than 0.5 nM. In an embodiment, the Kd of the antibody (or fragment) for HVEM domain is 0.1 nM or stronger.

The term “K_{d}”, as used herein, is intended to refer to the dissociation constant of an antibody-antigen interaction. One way of determining the K_{d} or binding affinity of antibodies to HVEM is by measuring binding affinity of monofunctional Fab fragments of the antibody. (The affinity constant is the inverted dissociation constant). To obtain monofunctional Fab fragments, an antibody (for example, IgG) can be cleaved with papain or expressed recombinantly. The affinity of a fragment of an anti-human HVEM antibody can be determined, for example, by surface plasmon resonance (BIAcore3000™ surface plasmon resonance (SPR) system, BIAcore Inc., Piscataway N.J.). CM5 chips can be activated with N-ethyl-N′-(3-dimethylaminopropyl)-carbodiinide hydrochloride (EDC) and N-hydroxysuccinimide (NHS) according to the supplier's instructions. The antigen can be diluted into 10 mM sodium acetate pH 4.0 and injected over the activated chip at a concentration of 0.005 mg/mL. Using variable flow time across the individual chip channels, two ranges of antigen density can be achieved: 100-200 response units (RU) for detailed kinetic studies and 500-600 RU for screening assays. Serial dilutions (0.1-10× estimated K_{d}) of purified Fab samples are injected for 1 min at 100 microliters/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data to a 1:1 Langmuir binding model (Karlsson, R. Roos, H. Fagerstam, L. Petersson, B. (1994). Methods Enzymology 6. 99-110, the content of which is hereby incorporated in its entirety) using the BIA evaluation program. Equilibrium dissociation constant (K_{d}) values are calculated as k_{off}/kₒₙ. This protocol is suitable for use in determining binding affinity of an antibody or fragment to any antigen. Other protocols known in the art may also be used. For example, ELISA.

An epitope that “specifically binds” to an antibody or a polypeptide is a term well understood in the art, and methods to determine such specific or preferential binding are also well known in the art. A molecular entity is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding.

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

Depending on the amino acid sequences of the constant domains of their heavy chains, antibodies (immunoglobulins) can be assigned to different classes. The antibody or fragment can be, e.g., any of an IgG, IgD, IgE, IgA or IgM antibody or fragment thereof, respectively. In an embodiment the antibody is an immunoglobulin G. In an embodiment the antibody fragment is a fragment of an immunoglobulin G. In an embodiment the antibody is an IgG1, IgG2, IgG2a, IgG2b, IgG3 or IgG4. In an embodiment the antibody comprises sequences from a human IgG1, human IgG2, human IgG2a, human IgG2b, human IgG3 or human IgG4. A combination of any of these antibodies subtypes can also be used. One consideration in selecting the type of antibody to be used is the desired serum half-life of the antibody. For example, an IgG generally has a serum half-life of 23 days, IgA 6 days, IgM 5 days, IgD 3 days, and IgE 2 days. (Abbas AK, Lichtman AH, Pober JS. Cellular and Molecular Immunology, 4th edition, W.B. Saunders Co., Philadelphia, 2000, hereby incorporated by reference in its entirety).

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) (or CDRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of an antibody (immunoglobulin) from any vertebrate species can be assigned to one of two clearly distinct types, called kappa (κ) and lambda (λ), based on the amino acid sequences of their constant domains.

“Framework” or “FR” residues are those variable domain residues other than the HVR residues as herein defined.

The term “hypervariable region” or “HVR” when used herein refers to the regions of an antibody variable domain which are hypervariable in sequence and/or form structurally defined loops. Generally, antibodies comprise six HVRs; three in the VH (H1, H2, H3) and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996). A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991) hereby incorporated by reference in its entirety). There are CDRs 1, 2, and 3 for each of the heavy and light chains. Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine of the Fc region may be removed, for example, during production or purification of the antibody, or by recombinantly engineering the nucleic acid encoding a heavy chain of the antibody. Accordingly, an intact antibody as used herein may be an antibody with or without the otherwise C-terminal lysine.

Compositions or pharmaceutical compositions comprising the antibody, scFv or fragment(s) of antibody disclosed herein can comprise stabilizers to prevent loss of activity or structural integrity of the protein due to the effects of denaturation, oxidation or aggregation over a period of time during storage and transportation prior to use. The compositions or pharmaceutical compositions can comprise one or more of any combination of salts, surfactants, pH and tonicity agents such as sugars can contribute to overcoming aggregation problems. Where a composition or pharmaceutical composition is used as an injection, a pH value is in an approximately neutral pH range, and it is advantageous to minimize surfactant levels to avoid bubbles in the formulation which are detrimental for injection into subjects. In an embodiment, the composition or pharmaceutical composition is in liquid form and stably supports a high concentration of bioactive antibody in solution, and is suitable for inhalational or parenteral administration. In an embodiment, the composition or pharmaceutical composition is suitable for intravenous, intramuscular, intraperitoneal, intradermal and/or subcutaneous injection. In an embodiment, the composition or pharmaceutical composition is in liquid form and has minimized risk of bubble formation and anaphylactoid side effects. In an embodiment, the composition or pharmaceutical composition is isotonic. In an embodiment, the composition or pharmaceutical composition has a pH or 6.8 to 7.4.

In an embodiment the scFv or fragment(s) of antibody disclosed herein are lyophilized and/or freeze dried and are reconstituted for use.

Examples of pharmaceutically acceptable carriers include, but are not limited to, phosphate buffered saline solution, sterile water (including water for injection USP), an emulsion such as an oil/water emulsion, and various types of wetting agents. A diluent for aerosol or parenteral administration includes phosphate buffered saline or normal (0.9%) saline, for example 0.9% sodium chloride solution, USP. Compositions comprising such carriers are formulated by methods known to those of skill in the art (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, the content of each of which is hereby incorporated in its entirety). In non-limiting examples, the carrier can comprise one or more of dibasic sodium phosphate, potassium chloride, monobasic potassium phosphate, polysorbate 80 (e.g. 2-[2-[3,5-bis(2-hydroxyethoxy)oxolan-2-yl]-2-(2-hydroxyethoxy)ethoxy]ethyl (E)-octadec-9-enoate), disodium edetate dehydrate, sucrose, monobasic sodium phosphate monohydrate, or dibasic sodium phosphate dihydrate.

The antibody, antibody fragments, compositions, or pharmaceutical composition described herein can also be lyophilized or provided in any suitable form including, but not limited to, an injectable solution, an inhalable solution, a gel form, and a tablet.

The term “Fc domain” as used herein is used to define a C-terminal region of an immunoglobulin heavy chain, including native sequence Fc regions and variant Fc regions. Although the boundaries of the Fc domain of an immunoglobulin heavy chain might vary, the human IgG heavy chain Fc domain is usually defined to stretch from an amino acid residue at position Cys226, or from Pro230, to the carboxyl-terminus thereof. The C-terminal lysine of the Fc domain may be removed, for example, by recombinantly engineering the nucleic acid encoding it. In embodiments, the antibody comprises an Fc domain. In an embodiment, the Fc domain has the same sequence or 99% or greater sequence similarity with a human IgG1 Fc domain. In an embodiment, the Fc domain has the same sequence or 99% or greater sequence similarity with a human IgG2 Fc domain. In an embodiment, the Fc domain has the same sequence or 99% or greater sequence similarity with a human IgG3 Fc domain. In an embodiment, the Fc domain has the same sequence or 99% or greater sequence similarity with a human IgG4 Fc domain. In an embodiment, the Fc domain is not mutated. In an embodiment, the Fc domain is mutated at the CH2 CH3 domain interface to increase the affinity of IgG for FcRn at acidic but not neutral pH (Dall'Acqua et al, 2006; Yeung et al, 2009). In an embodiment, the Fc domain has the same sequence as a human IgG1 Fc domain.

Amino acid sequence insertions include amino-and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N-or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but framework alterations are also contemplated. Conservative substitutions are shown in Table 1 under the heading of “conservative substitutions.” If such substitutions result in a change in biological activity, then more substantial changes, denominated “exemplary substitutions” in Table 1, or as further described below in reference to amino acid classes, may be introduced and the products screened.

TABLE 1
Amino Acid Substitutions

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

Substantial modifications in the biological properties of the antibody are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical. Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

In an embodiment, an antibody described herein is recombinantly produced. In an embodiment, the fusion protein is produced in a eukaryotic expression system.

In an embodiment, the fusion protein produced in the eukaryotic expression system comprises glycosylation at a residue on the Fc portion corresponding to Asn297.

In an embodiment the composition or pharmaceutical composition comprising the antibody, or antigen-binding fragment thereof, described herein is substantially pure with regard to the antibody, or antigen-binding fragment thereof. A composition or pharmaceutical composition comprising the antibody, or antigen-binding fragment thereof, described herein is “substantially pure” with regard to the antibody or fragment when at least 60% to 75% of a sample of the composition or pharmaceutical composition exhibits a single species of the antibody, or antigen-binding fragment thereof. A substantially pure composition or pharmaceutical composition comprising the antibody, or antigen-binding fragment thereof, described herein can comprise, in the portion thereof which is the antibody, or antigen-binding fragment, 60%, 70%, 80% or 90% of the antibody, or antigen-binding fragment, of the single species, more usually about 95%, and preferably over 99%. Purity or homogeneity may be tested by a number of means well known in the art, such as polyacrylamide gel electrophoresis or HPLC.

In an embodiment, the human HVEM has the protein sequence of SEQ ID NO:1 (GenBank: AAQ89238.1)

“And/or” as used herein, for example, with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “At least one” is not to be construed as limiting “a” or “an.” “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein

Throughout this application various publications are referred to. Full citations for these references may be found at the end of the specification. The disclosures of all publications, patents, patent application publications and books referred to herein, are hereby incorporated by reference in their entirety into the subject application to more fully describe the art to which the subject invention pertains.

This invention may be better understood from the Experimental Details, which follow.

### EXPERIMENTAL DETAILS

HVEM signaling has not been previously linked to the functionality of antibody responses. Viewing the unexpected observation that recombinant HSV-2 having a deletion of the entire HSV-2 glycoprotein D-encoding gene in the genome thereof (referred to herein as “ΔgD-2”) elicits predominantly ADCC responses, whereas natural infections and gD-expressing vaccines elicit neutralizing responses, it was hypothesized that HVEM signaling may promote the generation of ADCC and/or that gD may block this response. To test this hypothesis, we compared the immunogenicity and efficacy of ΔgD-2, d1529 (expresses gD) and recombinant gD protein vaccines in wild-type and HVEM, BTLA or LIGHT knockout mice. Results indicate that Fc gamma receptor IV (FcγRIV) ADCC mediating antibodies provide a strong correlate of immune protection against clinical isolates of HSV-1 and HSV-2 and that HVEM-LIGHT signaling is involved in this protective immune response. Moreover, in the absence of HVEM, or in the presence of gD, the ability of immune cells to trigger ADCC is impaired.

ΔgD and rgD-2 Differentially Protect From Infection With Clinical Isolates of HSV

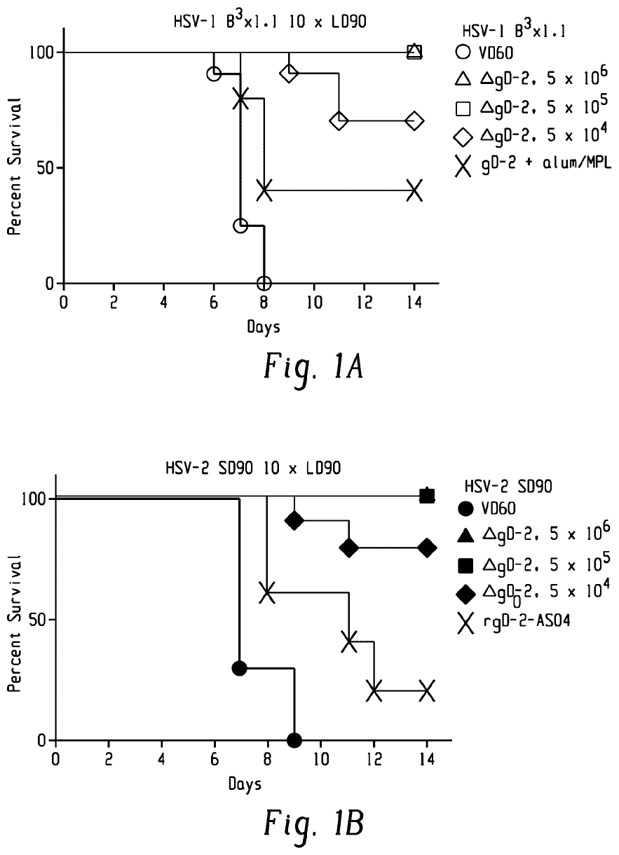

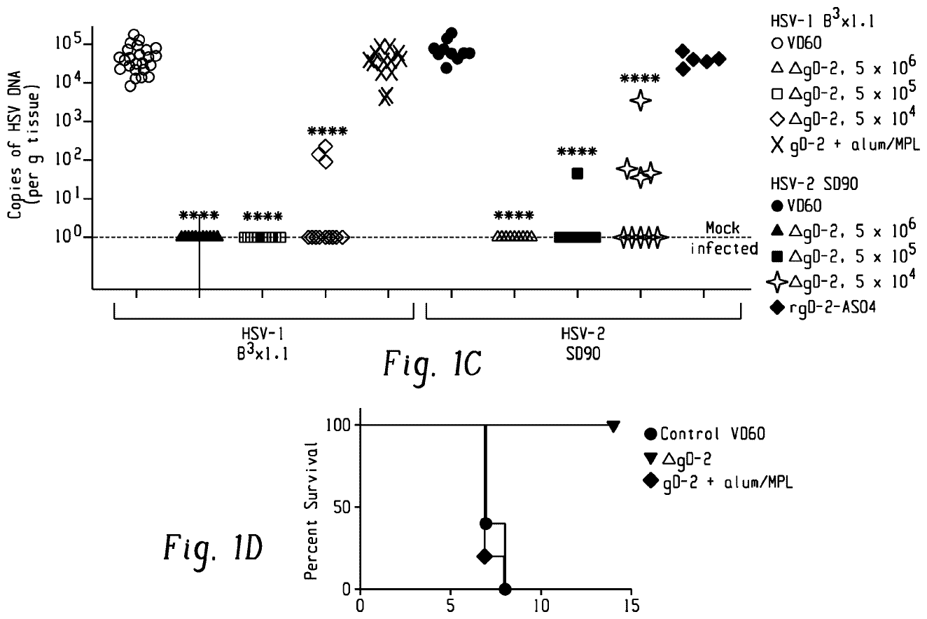

Candidate vaccines were shown to differ in immunogenicity and efficacy. To compare protection by ΔgD-2 and a rgD-2 vaccine, C57BL/6 mice were prime-boost vaccinated 3 weeks apart, with 5×10⁶, 5×10⁵ or 5×10⁴ pfu of ΔgD-2 or with 5 μg of rgD-2/AS04 (GlaxoSmithKline) or rgD-2/Alum-MPL. Three weeks following boost vaccination, mice were challenged on the skin with a 10× lethal dose (LD90) of HSV-1 (strain B³x1.1) (FIG. 1A) or HSV-2 (SD90) (FIG. 1B). ΔgD-2 protected 100% of mice at vaccine doses of 5×10⁶ or 5×10⁵ and protected 80% (HSV-1) and 70% (HSV-2) of mice following immunization with 5×10⁴ pfu/mouse. The rgD-2 protein vaccines provided limited protection against these high dose clinical isolates of HSV-1 (40%) or HSV-2 (20%). HSV DNA in dorsal root ganglia (DRG) was quantified as a marker of latency and the results paralleled the survival data (FIG. 1C). Viral DNA was only detected in 1 out of 10 mice vaccinated with the two highest doses of ΔgD-2 and even at the lowest vaccine dose, protection against latency was observed (DNA detected in 3/10 (HSV-1) and 4/10 (HSV-2) of mice). Passive transfer protection was assessed by transferring 750 μg of total IgG from immunized to naïve mice one day before HSV challenge. Only serum IgG from ΔgD-2-immunized mice was able to completely passively protect naïve mice from skin challenge with HSV-2 4674 (FIG. 1D), while rgD-2 with alum and MPL provided no protection.

ΔgD-2 (ΔgD) and rgD-2 Induce Functionally Different Antibody Responses to HSV

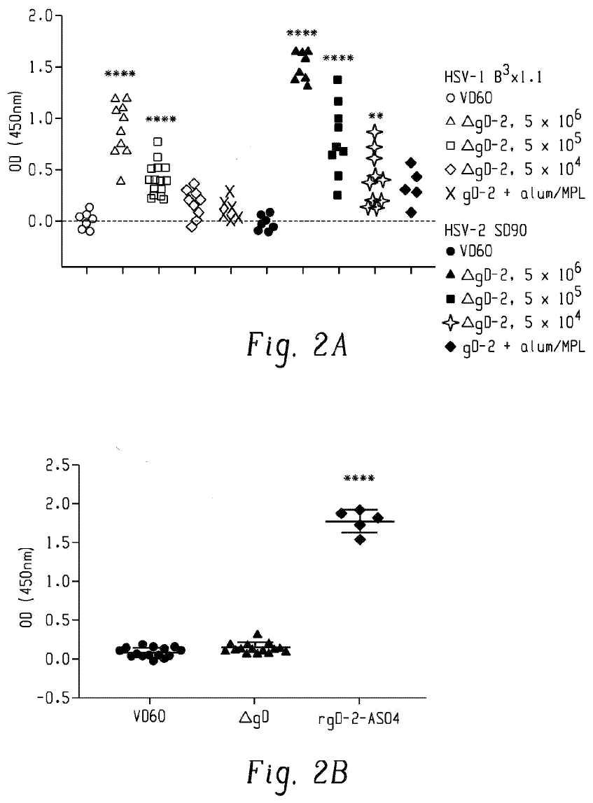

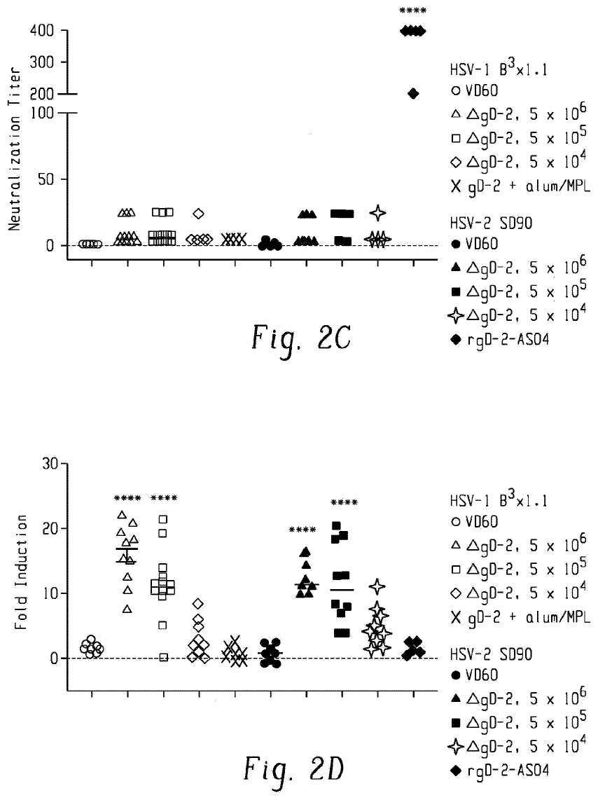

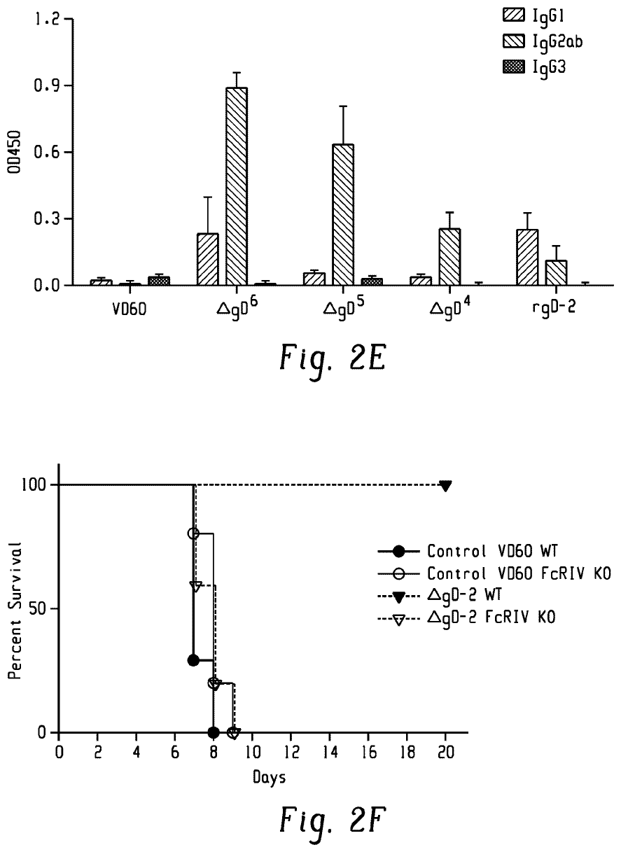

To determine whether the differences in efficacy were associated with the quantity and/or functionality of antibody responses, mice were immunized with 5×10⁴ pfu of ΔgD-2 or 5 μg rgD-2, and one week after boost vaccination, serum samples were collected and total HSV-2 specific IgG titer (1:90 000 dilution; FIG. 2A) or total gD-2 binding IgG (FIG. 2B) were quantified by ELISA. Response to HSV-2 was focused on as there was little serotype difference in efficacy and the vaccines are based on HSV-2 viruses and proteins. ΔgD-2 induced the highest total HSV-2 ELISA antibody titers, whereas rgD-2/AS04 induced the most robust gD-2 specific response; there was no gD-specific antibody detected in response to ΔgD-2 immunization. The functionality of the Ab responses also differed. Recombinant gD-2/AS04 induced the highest neutralizing titer (FIG. 2C), but little or no FcγRIV activation (FIG. 2D). Conversely, ΔgD-2 induced the most potent FcγRIV response, but little neutralizing activity. The differences in function were reflected by the relative proportion of HSV-2 specific IgG1 and IgG2 response. In mice, IgG2 is the isotypes most strongly associated with activation of FcγRIV and IgG1 with neutralizing antibodies (Nimmerjahn et al., 2005; Huber et al., 2006). ΔgD-2 induced a predominant IgG2 response, rgD-2/AS04 generated a predominant IgG1 response and d1529 elicited approximately equal proportions of IgG1 and IgG2 (FIG. 2E).

To further assess the link between FcγRIV activating antibodies and protection, passive transfer studies in FcγRIV−/− were conducted. Intraperitoneal administration of 750 μg serum isolated from ΔgD-2 immunized mice was transferred to naïve WT or FcγRIV−/− (FcRIV KO) mice. The serum isolated from the ΔgD-2 immunized mice completely protected naïve WT, but not FcγRIV−/− mice, whereas serum from control (VD60) vaccinated animals failed to protect naïve WT mice (FIG. 2F).

Vaccination of HVEM−/− Mice With ΔgD or d15-29 Abrogates/Reduces Protection

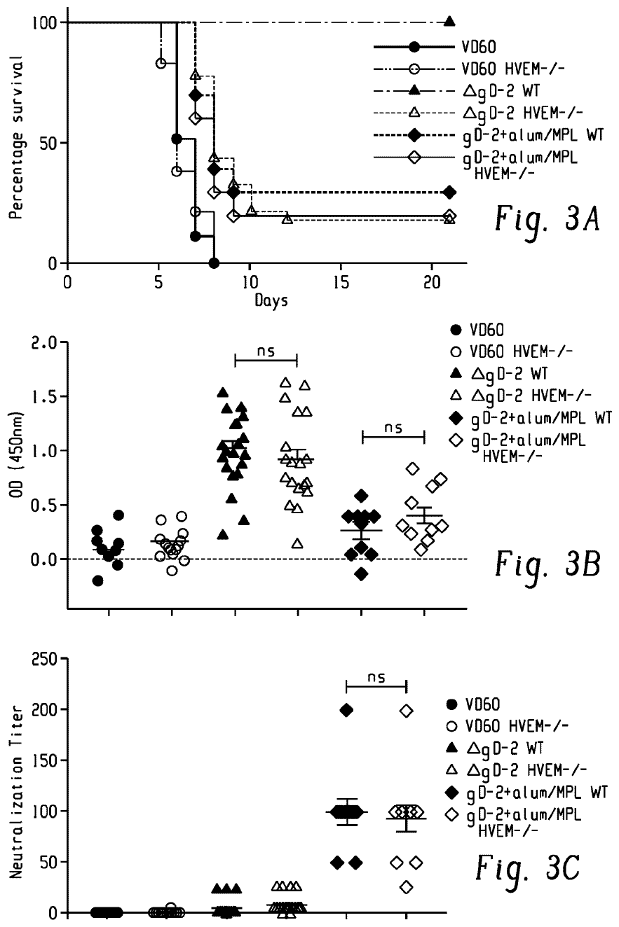

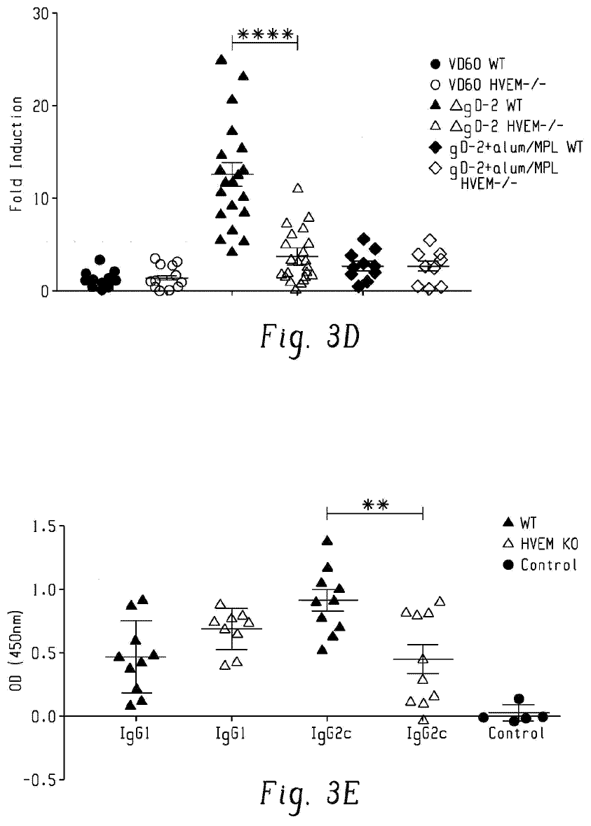

To explore the possibility that the shift from neutralizing to FcγRIV activating antibodies reflects the absence of gD blockade of HVEM interactions with one or more of its ligands, HVEM−/− mice were vaccinated with rgD-2/Alum-MPL or ΔgD-2 and then challenged with 10×LD₉₀ of strain SD90. There was a significant loss in protection in the HVEM−/− mice compared to WT mice in response to ΔgD-2 (p<0.0001), but no effect on the low level of protection observed with rgD-2/Alum-MPL (FIG. 3A). There was no significant decrease in total HSV-binding IgG (FIG. 3B) or neutralization responses (FIG. 3C), but a significant decrease in FcγRIV activation (FIG. 3D) and a decline in the proportion of IgG2 responses to ΔgD-2 (FIG. 3E).

### HVEM is Involved in Mounting and Mediating ADCC Effector Cell Response

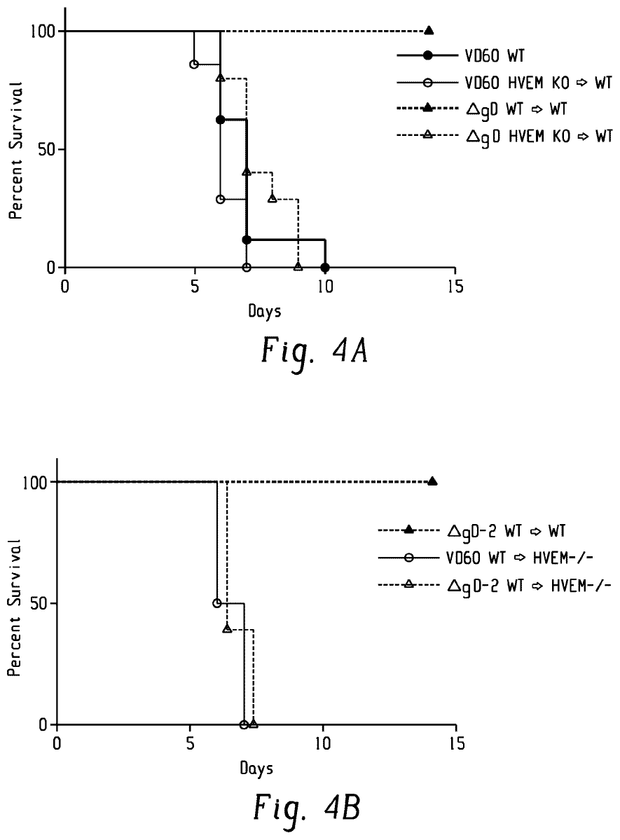

To test whether the reduction in IgG2, FcγRIV-activating antibodies elicited by ΔgD-2 in HVEM−/− (or LIGHT−/−) mice translates into a loss in ability to passively transfer protection to naïve WT mice, 750 μg of serum isolated from ΔgD-2 or VD60 (control) immunized WT or HVEM−/− mice, was administered intraperitoneally into WT or HVEM−/− mice one day prior to challenge with 10×LD₉₀ of strain SD90. Consistent with previous studies, ΔgD-2 immune serum from WT mice fully protected naïve WT mice and, consistent with the loss of FcγRIV-activating Abs, immune serum from HVEM−/− mice failed to protect. (FIG. 4A). However, unexpectedly, no protection was observed when ΔgD-2 immune serum from vaccinated WT mice was transferred into HVEM−/− mice (FIG. 4B), suggesting that HVEM signaling is required both for generating and mediating ADCC responses.

### BTLA Ligands Are Not Involved

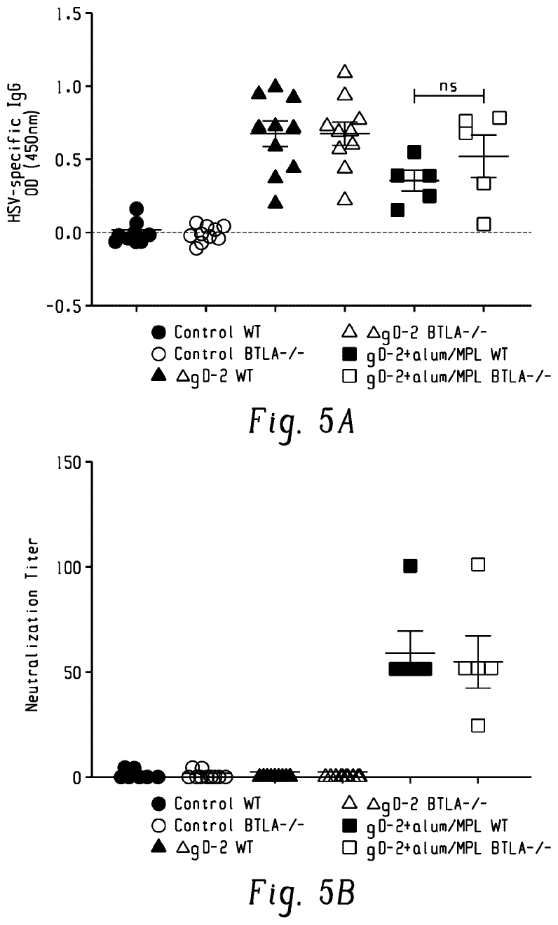

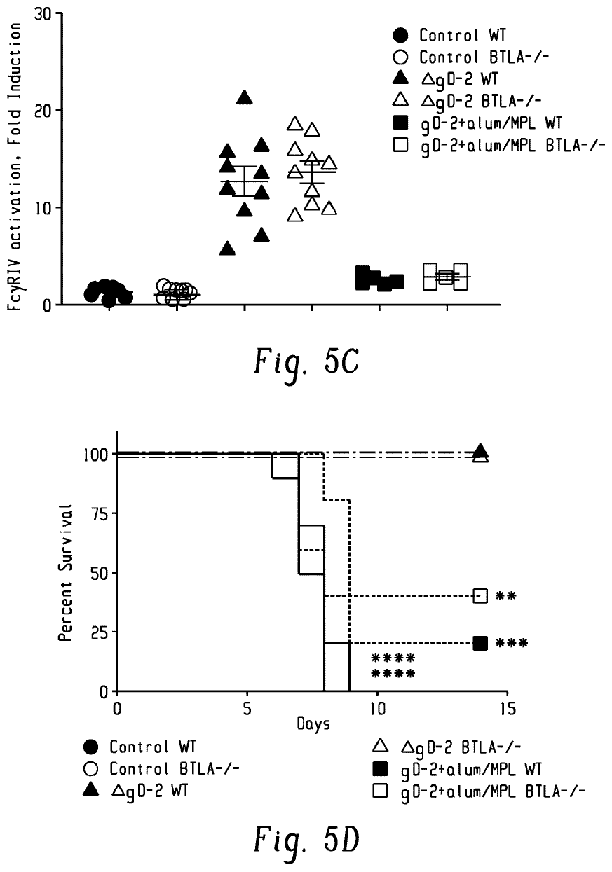

To explore the effect of BTLA on the generation of FcγRIV activating antibodies, WT (C57B1/6) mice or BTLA-/-mice were prime-boost vaccinated (3 week interval) with 5×10⁵ pfu ΔgD-2, VD-60 cell lysate (control) or 5 μg rgD-2 with alum and MPL. One week following boost vaccination, serum was obtained by retroorbital bleed and mice were challenged with 10×LD₉₀ of strain SD90. There was no significant difference in total HSV-binding IgG (FIG. 5A), neutralization responses (FIG. 5B), or FcγRIV activation (FIG. 5C). There was also no significant loss in protection in the BTLA−/− mice compared to WT mice in response to ΔgD-2 (FIG. 5D), suggesting that BTLA does not play a significant role in the generation of FcγRIV activating antibodies.

### Role of LIGHT Ligands

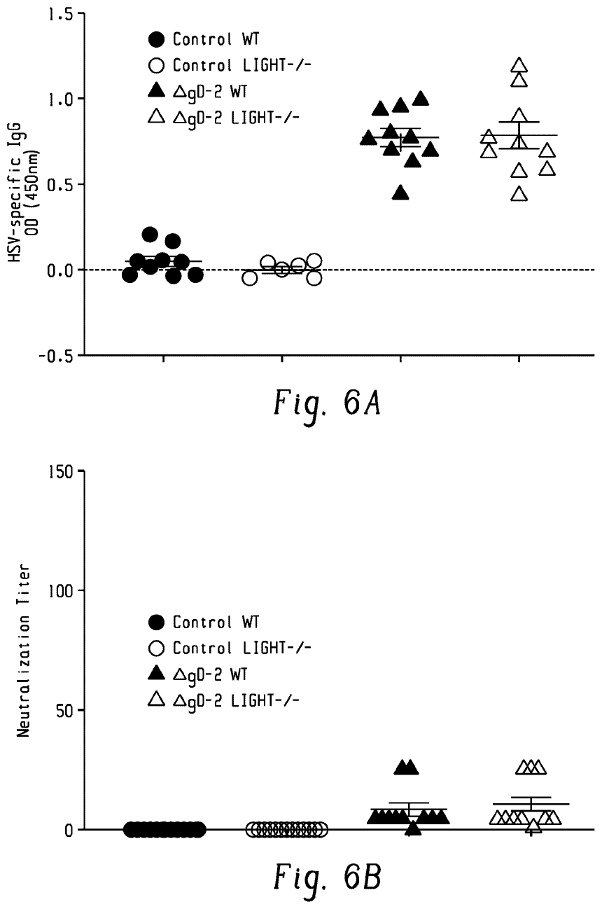

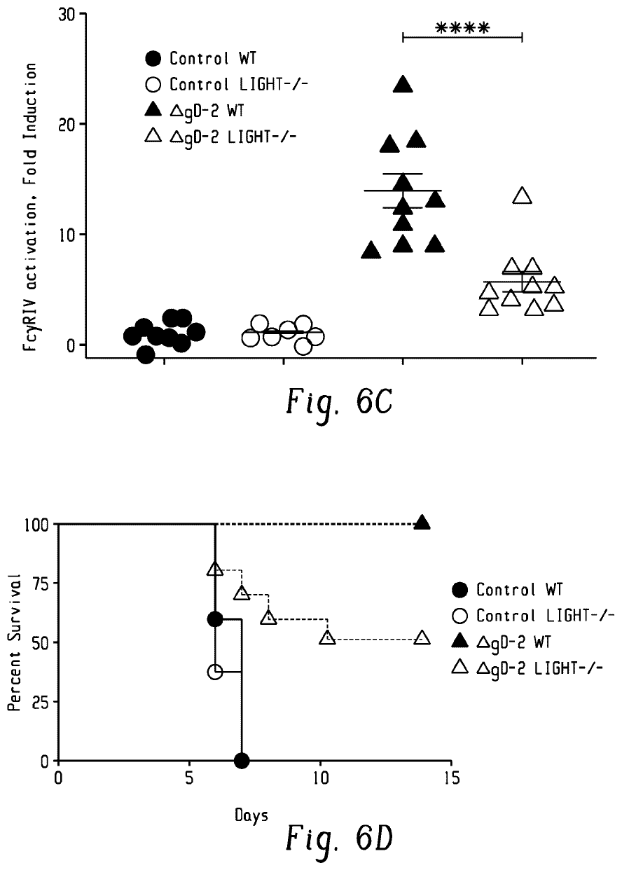

To explore the effect of LIGHT on the generation of FcγRIV activating antibodies, WT (C57B1/6) mice or LIGHT−/− mice were prime-boost vaccinated (3 week interval) with 5×10⁵ pfu ΔgD-2 or 5 VD-60 cell lysate (control). One week following boost vaccination, serum was obtained by retroorbital bleed and mice were challenged with 10×LD₉₀ of HSV-2 strain SD90. There was no significant difference in total HSV-binding IgG (FIG. 6A) or neutralization responses (FIG. 6B), however there was a significant decrease in FcγRIV activation in LIGHT−/− mice (FIG. 6C). Partial protection of LIGHT−/− mice was observed following infection with HSV-2 (FIG. 6D). These results suggest that interactions between HVEM and LIGHT play a role in the generation of FcγRIV activating antibodies, but that other ligands may also contribute.

### Cells Derived From HVEM−/− Mice are Deficient in Effector Function

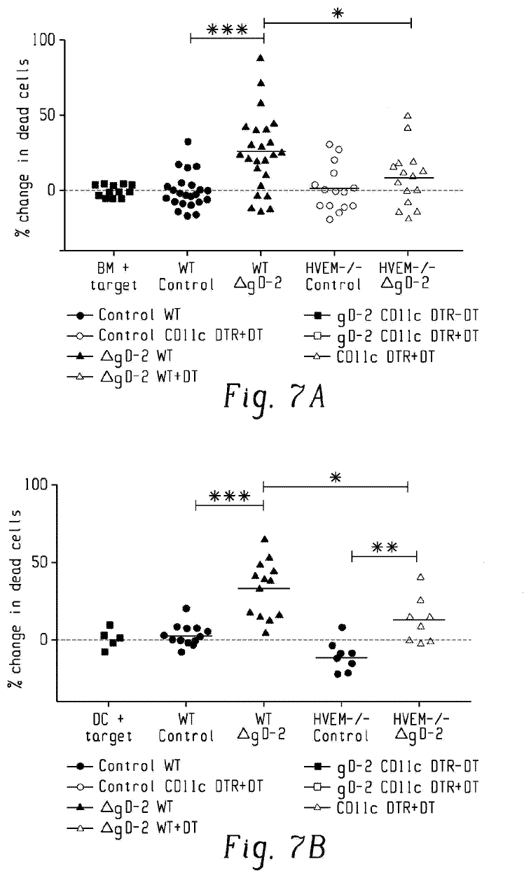

To further evaluate the role of HVEM in mediating ADCC, an ex vivo assay was conducted with WT or HVEM−/− whole bone marrow or different cell subpopulations as the effector cells and HSV-2-infected HaCAT cells as the target cells. Targets cells were infected with a strain of HSV-2 expressing GFP to identify infected cells, and cell killing was measured by the change in the percentage of dead, HSV-2 infected target cells following incubation with effector cells. Briefly, HaCAT cells were infected with HSV-2 333-ZAG (GFP) for 4 hours. Infected target cells were then incubated with immune serum from ΔgD-vaccinated mice (1:5 dilution) and total bone marrow or bone marrow-derived DCs from WT or HVEM−/− mice were used as effector cells. Killing by effector cells was measured by quantifying dead GFP+infected target cells by flow cytometry. Compared to cells isolated from WT mice, there was a significant reduction in ADCC when the effector cells were isolated from HVEM−/− bone marrow (See FIGS. 7A, 7B).

gD and anti-HVEM Antibodies Inhibit FcγRIV Activation

Serum from mice immunized with ΔgD-2 or VD-60 (control) were added, alone or in combination with recombinant gD, recombinant HSV glycoprotein B (gB), or anti-HVEM antibody, to ΔgD-2 infected Vero target cells and effector cells expressing mFcRIV linked to

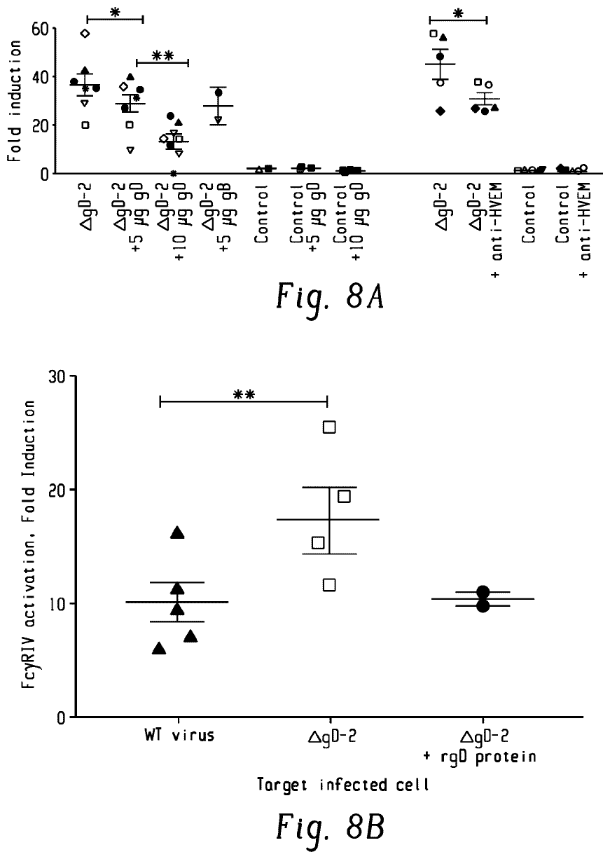

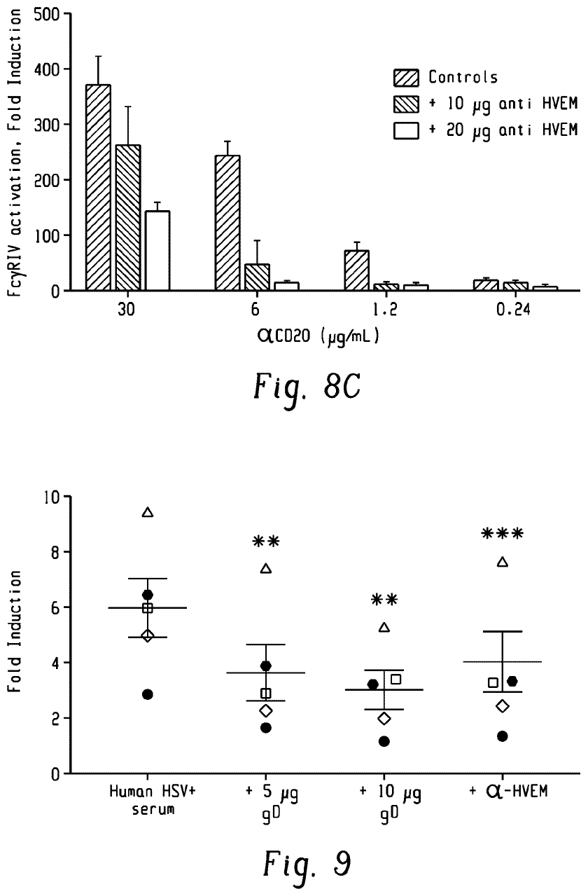

NFAT luciferase reporter (PROMEGA). As shown in FIG. 8A, soluble gD or anti-HVEM antibody reduces FcγRIV activation. When the serum from mice immunized with ΔgD-2 was combined with target cells infected with either WT HSV-2 or ΔgD-2 (i.e., no gD expression on infected cell), in the absence of gD in the target, FcγRIV activation is enhanced, however, the addition of recombinant gD protein brings the FcγRIV activation back down (FIG. 8B).

Instead of using immune serum, the assay was performed using Raji cells (express CD20) and rituximab (anti-CD20) in the absence or presence of anti-HVEM antibody (10 or 20 μg). As shown in FIG. 8C, anti-HVEM antibody inhibits FcγRIV activation indicating that HVEM is needed for optimal effector killing function.

### gD and Anti-HVEM Inhibit FcγRIIIa Activation by Human Serum

Human serum from five HSV seropositive individuals was incubated with HSV-2 (SD90) infected target cells and effector cells expressing human FcγRIIIa linked to NFAT luciferase reporter (PROMEGA) either alone or in combination with gD protein (5 μg or 10 μg) or anti-HVEM antibody (10 μg). As shown in FIG. 9, the human serum has low levels (5-6-fold) of FcγRIIIa activation antibodies (detected using a human kit to measure receptor activation of the human equivalent of m FcγRIV), and killing of cells is decreased when recombinant gD protein or anti-HVEM antibody is added.

### HVEM is Needed for Generating ADCC Abs Against Other Pathogens

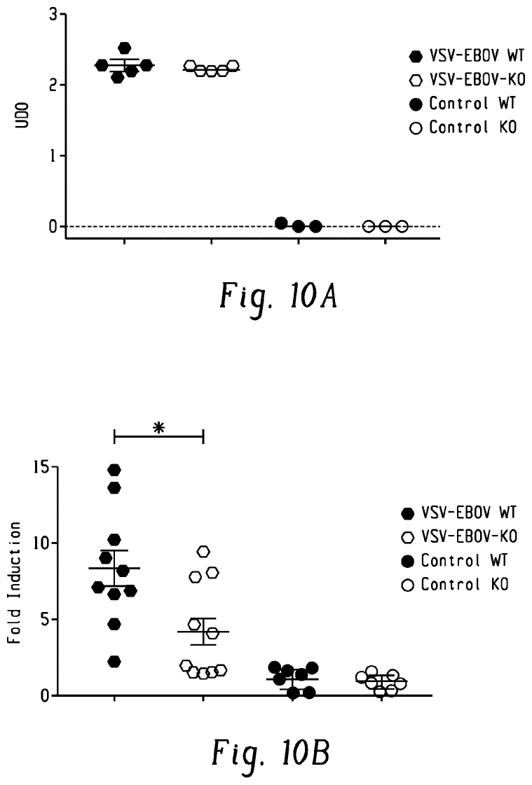

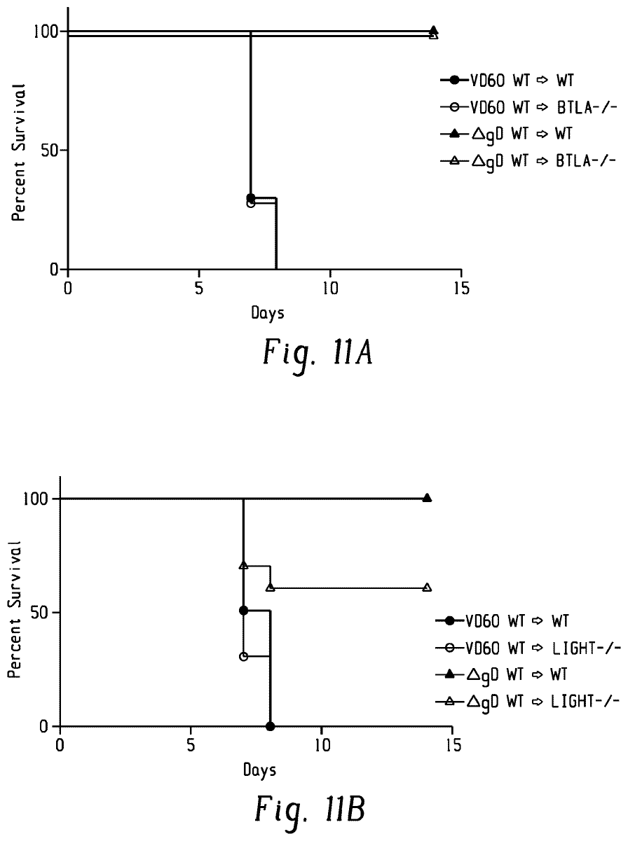

In this study, wild-type or HVEM−/− knockout mice were prime-boost immunized at 3 week intervals with 10⁴ pfu of a VSV-Ebola virus construct (Chandran lab) or with PBS (control) and the immune serum assayed for total antibody and ADCC activating Abs. Total serum antibody to the Ebola protein was measured, and the results are shown in FIG. 10A. Serum was incubated with VSV-Ebola infected target cells and effector cells expressing mFcγRIV linked to NFAT luciferase reporter (PROMEGA). As shown in FIG. 10B, there was a decrease in FcγRIV activating Abs in HVEM KO mice.

### Passive Transfer Shows Role for LIGHT Not BTLA

Serum from control mice immunized with VD60 or serum from mice immunized with ΔgD-2 was administered intraperitoneally into WT, BTLA−/−, or LIGHT−/− mice one day prior to challenge. As shown in FIGS. 11A and 11B, passive transfer of serum from mice immunized with ΔgD-2 was able to prevent mortality in BTLA−/− mice and but was less effective in LIGHT−/− mice.

### DISCUSSION

In contrast to other HSV vaccine efforts, which were designed to elicit neutralizing gD-targeted antibodies, ΔgD-2 induces a robust non-gD, FcγRIV-activating, ADCC response (Petro et al., 2015; 2016; Burn et al., 2017). This functional difference translates into greater protection in mice challenged with clinical isolates of HSV-1 or HSV-2. Although adjuvanted rgD-2 subunit formulations have been shown to elicit the highest gD-specific and neutralizing antibody response, these formulations provided only modest protection against primary disease and failed to prevent latency when mice were challenged with these clinical isolates. These results, coupled with the absence of passive protection by rgD-2 and loss of passive protection in FcγRIV knockout mice, indicate that ADCC responses provide a better correlate of immune protection. The central role of ADCC in mediating protection is supported by clinical studies. For example, HSV infected neonates who had disease limited to the skin acquired higher titers of ADCC antibodies from their mothers compared to infants who presented with disseminated disease (Kohl et al., 1989; Kohl, 1991).

The differential response to rgD-2 (IgG1 dominant, neutralizing) and ΔgD-2 (IgG2 dominant, FcγRIV activating) suggests that gD may block the generation of the more protective ADCC responses as an immune evasion strategy. While precisely what determines the type of antibody response to pathogens has not been fully elucidated, it was hypothesized that HVEM may play a “regulatory” role since gD competes with several HVEM ligands. Results of the current studies support this hypothesis. There was a reduction in FcγR activation, ADCC, and the proportion of IgG2 antibody responses to ΔgD-2 in HVEM knockout compared to WT mice. Similar results were observed with LIGHT−/−, but not BTLA−/− mice suggesting that HVEM-LIGHT signaling promotes the generation of ADCC responses. Notably, there was no decrease in the total HSV-binding antibody responses to ΔgD-2 or in the neutralization response to rgD-2 protein vaccine in the HVEM−/− mice, suggesting that HVEM signaling contributes specifically to the generation of ADCC responses, but is not required for eliciting antibody responses in general. Other gD-expressing vaccines and natural infection preferentially generate neutralizing antibodies with variable amounts of ADCC responses and we propose that this reflects the ability of virally produced gD to block HVEM-LIGHT signaling. The variable ADCC antibody response to natural infection may reflect individual differences in HVEM expression and signaling.

Notably, HVEM signaling was required not only for mounting an FcR-activating antibody response, but also for mediating ADCC. This conclusion is supported by passive transfer studies. Intraperitoneal administration of serum collected from ΔgD-2 immunized mice completely protected WT but not HVEM−/− mice against subsequent viral challenge. A role for HVEM in mediating antibody dependent cell killing was confirmed by in vitro studies in which effector cells derived from HVEM−/− mice were substantially less able to kill HSV-infected target cells compared to cells from WT mice.

The ability of gD-2 protein to interfere with effector function was illustrated by a dose-dependent block of FcγRIV activation in the presence of gD-2. Conversely, FcγRIV activation was increased if the target cells were infected with ΔgD-2 rather than WT virus and, thus, did not express gD on the plasma membrane, indicating a corollary immune evasion strategy. Glycoprotein D competes with HVEM signaling to block the generation of ADCC antibody responses but, even if the antibodies are present, interferes with effector cell killing. The results thus point to the use of glycoprotein D as an HVEM antagonist to minimize and/or block ADCC response in conditions where ADCC has a detrimental effect, for example, in autoimmune disease.

Interfering with HVEM signaling to block the generation of ADCC responses and/or the ability of effector cells to induce killing might be particularly relevant for pathogens that escape neutralizing antibodies. For example, the cytomegalovirus (CMV) UL144 protein is an orthologue of HVEM that targets BTLA (Cheung et al., 2005). The function of this protein in CMV pathogenesis is unknown, but it has been hypothesized as having a role in immune evasion (Poole et al., 2006).

The results from this study undercover a previously unrecognized regulatory role for HVEM signaling in generating and effecting antibody dependent cell mediating killing responses and identifies a novel HSV immune evasion strategy. By competing with LIGHT for HVEM binding, gD interferes with the generation of the FcγRIV activating antibodies and, even if these antibodies are produced, interferes with their ability to mediate target cell killing. This may contribute to the preferential induction of neutralizing antibody responses to natural infections, as viruses can escape these antibodies by spreading directly from cell-to-cell.

Set forth below are some embodiments of the methods disclosed herein.

Embodiment 1: A method of preferentially enhancing in a subject an antibody-dependent cell-mediated cytotoxicity (ADCC) antibody response to a vaccine for an infectious agent, comprising administering to the subject receiving the vaccine an amount of an herpesvirus entry mediator (HVEM) agonist, a tumor necrosis factor superfamily-14 (TNFSF-14) agonist, or a combination thereof, effective to enhance an ADCC antibody response in the subject.

Embodiment 2: A method of enhancing antibody-dependent cell-mediated cytotoxicity (ADCC) activity of a vaccine for an infectious agent that elicits a neutralizing antibody response, comprising administering to the subject receiving the vaccine for an infectious agent an amount of an herpesvirus entry mediator (HVEM) agonist, a tumor necrosis factor superfamily-14 (TNFSF-14) agonist, or a combination thereof, effective to enhance an ADCC activity in the subject.

Embodiment 3: The method of Embodiment 1 or Embodiment 2, wherein the HVEM agonist comprises a tumor necrosis factor superfamily-14 (TNFSF-14) protein or a portion thereof.

Embodiment 4: The method of any one of Embodiments 1 to 3, wherein the HVEM agonist comprises a hexavalent TNFSF fusion protein.

Embodiment 5: The method of Embodiment 4, wherein the hexavalent TNFSF fusion protein comprises a single chain polypeptide comprising three TNFSF-14 subsequences folded into a functional trivalent receptor binding domain, and fused at a C-terminus thereof to a silenced IgG1 Fc-domain as a dimerization scaffold.

Embodiment 6: The method of any one of Embodiments 1-5, wherein the HVEM agonist is an agonist antibody which binds HVEM.

Embodiment 7: The method of any one of Embodiments 1 to 6, wherein the HVEM agonist is a single-chain variable fragment (scFv) of a monoclonal antibody which binds HVEM.

Embodiment 8: The method of any one of Embodiments 1 to 7, wherein the subject is not administered an additional immunostimulatory agent.

Embodiment 9: The method of any one of Embodiments 1 to 8, wherein the subject is not administered a TNFSF stimulatory agent, a TNFSF inhibitory agent, or a combination thereof.

Embodiment 10: The method of any one of Embodiments 1 to 9, wherein the subject is not administered a toll-like receptor (TLR) agonist, a CD40 agonist, a CD27 agonist, a MDA5 agonist, a nucleotide-binding oligomerization domain-containing protein (NOD), or a combination thereof.

Embodiment 11: The method of any one of Embodiments 1 to 10, wherein the subject is not administered a domain present in neuronal apoptosis inhibitory protein (NAIP), a domain present in a class II transactivator (CIITA), a domain present in hydroxyeicosatetraenoic acid (HET-E), a domain present in TP-1 (NACHT)-leucine rich repeat, a domain present in a nod-like receptor (NLR) agonist, an RIG-like helicase (RLH) agonist, a cytokine/chemokine receptor agonist, a purinergic receptor agonist, or a combination thereof.

Embodiment 12: The method of any one of Embodiments 1 to 11, wherein the vaccine is not a DNA vaccine or a genetic vaccine.

Embodiment 13: The method of any one of Embodiments 1 to 12, wherein the vaccine does not comprise a cancer vaccine, an anti-tumor vaccine, or a vaccine for a target on a tumor.

Embodiment 14: The method of any one of Embodiments 1 to 13, wherein the vaccine is a vaccine against an infectious agent.

Embodiment 15: The method of any one of Embodiments 1 to 14, wherein the vaccine is vaccine against a virus, a bacteria, or a combination thereof.

Embodiment 16: The method of any one of Embodiments 1 to 15, wherein the method elicits production of Fc gamma receptor IV-binding antibodies.

Embodiment 17: A method of preferentially enhancing in a subject an antibody-dependent cell-mediated cytotoxicity (ADCC) antibody response to a vaccine for an infectious agent, comprises administering to the subject receiving the vaccine an amount of tumor necrosis factor superfamily-14 (TNFSF) protein effective to enhance an ADCC antibody response.

Embodiment 18: A method of enhancing antibody-dependent cell-mediated cytotoxicity (ADCC) activity of a vaccine for an infectious agent that elicits a neutralizing antibody response, comprises administering to the subject receiving the vaccine for an infectious agent an amount of TNFSF-14 protein effective to enhance an ADCC activity in the subject.

Embodiment 19: A composition comprising a vaccine for an infectious agent and an amount of an herpesvirus entry mediator (HVEM) agonist effective to enhance an antibody-dependent cell-mediated cytotoxicity (ADCC) antibody response over a neutralizing antibody response.

Embodiment 20: The composition of Embodiment 19, wherein the HVEM agonist comprises a TNF superfamily (TNFSF) protein.

Embodiment 21: The composition of Embodiment 19 or Embodiment 0, wherein the HVEM agonist is a hexavalent TNFSF fusion protein.

Embodiment 22: The composition of any one of Embodiments 19-21, wherein the hexavalent TNFSF fusion protein comprises a single chain polypeptide comprising three TNFSF-14 subsequences folded into a functional trivalent receptor binding domain, fused at a C-terminus thereof to a silenced IgG1 Fc-domain as a dimerization scaffold.

Embodiment 23: The composition of any one of Embodiments 19-22, wherein the HVEM agonist is an agonist antibody which binds HVEM.

Embodiment 24: The composition of any one of Embodiments 19-23, wherein the HVEM agonist is a single-chain variable fragment (scFv) of a monoclonal antibody which binds HVEM.

Embodiment 25: A kit for enhancing a vaccine response, comprising:
(i) an amount of an HVEM agonist; and
(ii) an amount of a vaccine for an infectious agent.

Embodiment 26: The kit of Embodiment 25, wherein the HVEM agonist is a hexavalent tumor necrosis factor superfamily (TNFSF) fusion protein.

Embodiment 27: The kit of Embodiment 25 or Embodiment 26, wherein the TNFSF hexavalent fusion protein comprises a single chain polypeptide comprising three TNFSF-14 subsequences folded into a functional trivalent receptor binding domain, fused at a C-terminus thereof to a silenced IgG1 Fc-domain as a dimerization scaffold.

Embodiment 28: The kit of any one of Embodiments 25-27, wherein the HVEM agonist is an agonist antibody which binds HVEM.

Embodiment 29: The kit of any one of Embodiments 25-28, wherein the HVEM agonist is a single-chain variable fragment of an agonist monoclonal antibody which binds HVEM.

Embodiment 30: A method of decreasing or blocking Fc-gamma receptor (FcγR)-mediated killing of self-antigen in a subject having an autoimmune disease comprising administering to the subject an amount of an HVEM antagonist, a soluble herpes simplex virus (HSV) glycoprotein D, an antibody binding HVEM, or a combination thereof, effective to decrease or block the FcγR-mediated killing in the subject.

Embodiment 31: A method of blocking Fc-gamma receptor (FcγR)-mediated killing of self-antigen in a subject having an autoimmune disease comprising administering to the subject an amount of an HVEM antagonist, a soluble herpes simplex virus (HSV) glycoprotein D, an antibody binding HVEM, or a combination thereof, effective to reduce the autoimmune disease in the subject.

Embodiment 32: The method of Embodiment 30 or Embodiment 31, wherein the soluble HSV glycoprotein D comprises HSV-1 glycoprotein D, HSV-2 glycoprotein D, or a combination thereof.

### REFERENCES

ADDIN PAPERS2_CITATIONS <papers2_bibliography/> Awasthi, S., and H.M. Friedman. 2014. Status of prophylactic and therapeutic genital herpes vaccines. Current Opinion in Virology. 6:6-12. doi:10.1016/j.coviro.2014.02.006.
Belshe, R.B., P.A. Leone, D.I. Bernstein, A. Wald, M.J. Levin, J.T. Stapleton, I. Gorfinkel, R.L.A. Morrow, M.G. Ewell, A. Stokes-Riner, G. Dubin, T.C. Heineman, J.M. Schulte, C.D. Deal, Herpevac Trial for Women. 2012. Efficacy results of a trial of a herpes simplex vaccine. N Engl J Med. 366:34-43. doi:10.1056/NEJMoa1103151.
Bernard, M.-C., V. Barban, F. Pradezynski, A. de Montfort, R. Ryall, C. Caillet, and P. Londoño-Hayes. 2015. Immunogenicity, Protective Efficacy, and Non-Replicative Status of the HSV-2 Vaccine Candidate HSV529 in Mice and Guinea Pigs. PLOS ONE. 10:e0121518-21. doi:10.1371/journal.pone.0121518.
Burn, C., N. Ramsey, S.J. Garforth, S. Almo, W.R. Jacobs Jr, and B.C. Herold. 2017. An HSV-2 single-cycle candidate vaccine deleted in glycoprotein D, ΔgD-2, protects male mice from lethal skin challenge with clinical isolates of HSV-1 and HSV-2. Journal of Infectious Diseases. 1-5. doi:10.1093/infdis/jix628.
Cai, G., A. Anumanthan, J.A. Brown, E.A. Greenfield, B. Zhu, and G.J. Freeman. 2008. CD160 inhibits activation of human CD4+ T cells through interaction with herpesvirus entry mediator. Nat. Immunol. 9:176-185. doi:10.1038/ni1554.
Cai, G., and G.J. Freeman. 2009. The CD160, BTLA, LIGHT/HVEM pathway: a bidirectional switch regulating T-cell activation. Immunological Reviews. 229:244-258. doi:10.1111/j.1600-065X.2009.00783.x.
Carfi, A., H. Gong, H. Lou, S.H. Willis, G.H. Cohen, R.J. Eisenberg, and D.C. Wiley. 2002. Crystallization and preliminary diffraction studies of the ectodomain of the envelope glycoprotein D from herpes simplex virus 1 alone and in complex with the ectodomain of the human receptor HveA. Acta Crystallogr. D Biol. Crystallogr. 58:836-838. doi:10.1107/S0907444902001270.
Carfi, A., S.H. Willis, J.C. Whitbeck, C. Krummenacher, G.H. Cohen, R.J. Eisenberg, and D.C. Wiley. 2001. Herpes simplex virus glycoprotein D bound to the human receptor HveA. Mol. Cell. 8:169-179.
Cheung, T.C., I.R. Humphreys, K.G. Potter, P.S. Norris, H.M. Shumway, B.R. Tran, G. Patterson, R. Jean-Jacques, M. Yoon, P.G. Spear, K.M. Murphy, N.S. Lurain, C.A. Benedict, and C.F. Ware. 2005. Evolutionarily divergent herpesviruses modulate T cell activation by targeting the herpesvirus entry mediator cosignaling pathway. Proc. Natl. Acad. Sci. U.S.A. 102:13218-13223. doi:10.1073/pnas.0506172102.
Cheung, T.C., M.W. Steinberg, L.M. Oborne, M.G. Macauley, S. Fukuyama, H. Sanjo, C. D'Souza, P.S. Norris, K. Pfeffer, K.M. Murphy, M. Kronenberg, P.G. Spear, and C.F. Ware. 2009. Unconventional ligand activation of herpesvirus entry mediator signals cell survival. Proc. Natl. Acad. Sci. U.S.A. 106:6244-6249. doi:10.1073/pnas.0902115106.
Compaan, D.M., L.C. Gonzalez, I. Tom, K.M. Loyet, D. Eaton, and S.G. Hymowitz. 2005. Attenuating lymphocyte activity: the crystal structure of the BTLA-HVEM complex. J. Biol. Chem. 280:39553-39561. doi:10.1074/jbc.M507629200.
Connolly, S.A., D.J. Landsburg, A. Carfi, D.C. Wiley, G.H. Cohen, and R.J. Eisenberg. 2003. Structure-based mutagenesis of herpes simplex virus glycoprotein D defines three critical regions at the gD-HveA/HVEM binding interface. J. Virol. 77:8127-8140. doi:10.1128/JVI.77.14.8127-8140.2003.
Connolly, S.A., D.J. Landsburg, A. Carfi, D.C. Wiley, R.J. Eisenberg, and G.H. Cohen. 2002. Structure-Based Analysis of the Herpes Simplex Virus Glycoprotein D Binding Site Present on Herpesvirus Entry Mediator HveA (HVEM). J. Virol. 76:10894-10904. doi:10.1128/JVI.76.21.10894-10904.2002.
Corey, L., and J.T. Schiffer. 2013. Rapid host immune response and viral dynamics in herpes simplex virus-2 infection. Nat. Med. 19:280-290. doi:10.1038/nm.3103.
Da Costa, X.J.E.A., C.A. Jones, and D.M. Knipe. 1999. Immunization against genital herpes with a vaccine virus that has defects in productive and latent infection. Proc. Natl. Acad. Sci. U.S.A. 96:6994-6998. doi:10.1086/605645.
Da Costa, X.J.E.A., L.A. Morrison, and D.M. Knipe. 2001. Comparison of Different Forms of Herpes Simplex Replication-Defective Mutant Viruses as Vaccines in a Mouse Model of HSV-2 Genital Infection. Virology. 288:256-263. doi:10.1006/viro.2001.1094.
Da Costa, X.J.E.A., M.F. Kramer, J. Zhu, M.A. Brockman, and D.M. Knipe. 2000. Construction, phenotypic analysis, and immunogenicity of a UL5/UL29 double deletion mutant of herpes simplex virus 2. J. Virol. 74:7963-7971.
De Trez, C., K. Schneider, K. Potter, N. Droin, J. Fulton, P.S. Norris, S.-W. Ha, Y.-X. Fu, T. Murphy, K.M. Murphy, K. Pfeffer, C.A. Benedict, and C.F. Ware. 2008. The inhibitory HVEM-BTLA pathway counter regulates lymphotoxin receptor signaling to achieve homeostasis of dendritic cells. The Journal of Immunology. 180:238-248.
del Rio, M.L., C.L. Lucas, L. Buhler, G. Rayat, and J.I. Rodriguez-Barbosa. 2010. HVEM/LIGHT/BTLA/CD160 cosignaling pathways as targets for immune regulation. Journal of Leukocyte Biology. 87:223-235. doi:10.1189/jlb.0809590.
Delagrave, S., H. Hernandez, C. Zhou, J.F. Hamberger, S.T. Mundle, J. Catalan, S. Baloglu, S.F. Anderson, J.M. DiNapoli, P. Londoño-Hayes, M. Parrington, J. Almond, and H. Kleanthous. 2012. Immunogenicity and Efficacy of Intramuscular Replication-Defective and Subunit Vaccines against Herpes Simplex Virus Type 2 in the Mouse Genital Model. PLOS ONE. 7:e46714-9. doi:10.1371/journal.pone.0046714.
Dingwell, K.S., and D.C. Johnson. 1998. The herpes simplex virus gE-gI complex facilitates cell-to-cell spread and binds to components of cell junctions. J. Virol. 72:8933-8942.
Dropulic, L., K. Wang, M. Oestreich, H.P., D. Garabedian, S. Jegaskanda, K. Dowdell, H. Nguyen, K. Laing, D. Koelle, A. Azose, S. Hunsberger, K. Lumbard, A. Chen, L.-J. Chang, S. Phogat, and J. Cohen. 2017. A Replication-Defective Herpes Simplex Virus (HSV)-2 Vaccine, HSV529, is Safe and Well-Tolerated in Adults with or without HSV Infection and Induces Significant HSV-2 Specific Antibody Responses in HSV Seronegative Individuals. OFID. S415-S416. doi:10.1093/ofid/ofx163.1041.
Dudek, T.E., E. Torres-Lopez, C. Crumpacker, and D.M. Knipe. 2011. Evidence for Differences in Immunologic and Pathogenesis Properties of Herpes Simplex Virus 2 Strains From the United States and South Africa. Journal of Infectious Diseases. 203:1434-1441. doi:10.1093/infdis/jir047.
Duhen, T., C. Pasero, F.O. Mallet, B. Barbarat, D. Olive, and R.G.T. Costello. 2004. LIGHT costimulates CD40 triggering and induces immunoglobulin secretion; a novel key partner in T cell-dependent B cell terminal differentiation. Eur. J. Immunol. 34:3534-3541. doi:10.1002/eji.200425598.
Harrop, J.A., M. Reddy, K. Dede, M. Brigham-Burke, S. Lyn, K.B. Tan, C. Silverman, C. Eichman, R. DiPrinzio, J. Spampanato, T. Porter, S. Holmes, P.R. Young, and A. Truneh. 1998a. Antibodies to TR2 (herpesvirus entry mediator), a new member of the TNF receptor superfamily, block T cell proliferation, expression of activation markers, and production of cytokines. The Journal of Immunology. 161:1786-1794.
Harrop, J.A., P.C. McDonnell, M. Brigham-Burke, S.D. Lyn, J. Minton, K.B. Tan, K. Dede, J. Spampanato, C. Silverman, P. Hensley, R. DiPrinzio, J.G. Emery, K. Deen, C. Eichman, M. Chabot-Fletcher, A. Truneh, and P.R. Young. 1998b. Herpesvirus entry mediator ligand (HVEM-L), a novel ligand for HVEM/TR2, stimulates proliferation of T cells and inhibits HT29 cell growth. J. Biol. Chem. 273:27548-27556.
Heo, S.-K., S.-A. Ju, S.-C. Lee, S.-M. Park, S.-Y. Choe, B. Kwon, B.S. Kwon, and B.-S. Kim. 2005. LIGHT enhances the bactericidal activity of human monocytes and neutrophils via HVEM. Journal of Leukocyte Biology. 79:330-338. doi:10.1189/jlb.1104694.
Hoshino, Y., L. Pesnicak, K.C. Dowdell, J. Lacayo, T. Dudek, D.M. Knipe, S.E. Straus, and J.I. Cohen. 2008. Comparison of immunogenicity and protective efficacy of genital herpes vaccine candidates herpes simplex virus 2 dl5-29 and d15-29-41L in mice and guinea pigs. Vaccine. 26:4034-4040. doi:10.1016/j.vaccine.2008.05.022.
Hoshino, Y., L. Pesnicak, K.C. Dowdell, P.D. Burbelo, D.M. Knipe, S.E. Straus, and J.I. Cohen. 2009. Protection from herpes simplex virus (HSV)-2 infection with replication-defective HSV-2 or glycoprotein D2 vaccines in HSV-1-seropositive and HSV-1-seronegative guinea pigs. Journal of Infectious Diseases. 200:1088-1095. doi: 10.1086/605645.
Hoshino, Y., S.K. Dalai, K. Wang, L. Pesnicak, T.Y. Lau, D.M. Knipe, J.I. Cohen, and S.E. Straus. 2004. Comparative Efficacy and Immunogenicity of Replication-Defective, Recombinant Glycoprotein, and DNA Vaccines for Herpes Simplex Virus 2 Infections in Mice and Guinea Pigs. J. Virol. 79:410-418. doi:10.1128/JVI.79.1.410-418.2005.
Huber, V.C., R.M. Mckeon, M.N. Brackin, L.A. Miller, R. Keating, S.A. Brown, N. Makarova, D.R. Perez, G.H. MacDonald, and J.A. McCullers. 2006. Distinct Contributions of Vaccine-Induced Immunoglobulin G1 (IgG1) and IgG2a Antibodies to Protective Immunity against Influenza. Clinical and Vaccine Immunology. 13:981-990. doi:10.1128/CVI.00156-06.
Johnson, D.C., and M.T. Huber. 2002. Directed egress of animal viruses promotes cell-to-cell spread. J. Virol. 76:1-8. doi:10.1128/JVI.76.1.1-8.2002.
Kohl, S. 1991. Role of antibody-dependent cellular cytotoxicity in defense against herpes simplex virus infections. Rev. Infect. Dis. 13:108-114.
Kohl, S., M.S. West, C.G. Prober, W.M. Sullender, L.S. Loo, and A.M. Arvin. 1989. Neonatal antibody-dependent cellular cytotoxic antibody levels are associated with the clinical presentation of neonatal herpes simplex virus infection. Journal of Infectious Diseases. 160:770-776.
Kwon, B.S., K.B. Tan, J. Ni, K.O. Oh, Z.H. Lee, K.K. Kim, Y.J. Kim, S. Wang, R. Gentz, G.L. Yu, J. Harrop, S.D. Lyn, C. Silverman, T.G. Porter, A. Truneh, and P.R. Young. 1997. A newly identified member of the tumor necrosis factor receptor superfamily with a wide tissue distribution and involvement in lymphocyte activation. J. Biol. Chem. 272:14272-14276.
Ligas, M.W., and D.C. Johnson. 1988. A herpes simplex virus mutant in which glycoprotein D sequences are replaced by beta-galactosidase sequences binds to but is unable to penetrate into cells. J. Virol. 62:1486-1494.
Looker, K.J., A.S. Magaret, K.M.E. Turner, P. Vickerman, S.L. Gottlieb, and L.M. Newman. 2015. Global Estimates of Prevalent and Incident Herpes Simplex Virus Type 2 Infections in 2012. PLOS ONE. 10:e114989-23. doi:10.1371/journal.pone.0114989.
Looker, K.J., J. Elmes, S.L. Gottlieb, and J.T. Schiffer. 2017. Effect of HSV-2 infection on subsequent HIV acquisition: an updated systematic review and meta-analysis. The Lancet Infectious . . . , doi:10.1016/S1473-3099 (17) 30405-X.
Manzanero, S. 2012. Generation of Mouse Bone Marrow-Derived Macrophages. In Leucocytes: Methods and Protocols. R.B. Ashman, editor. Humana Press, Totowa, NJ. 177-181.
Mauri, D.N., R. Ebner, R.I. Montgomery, K.D. Kochel, T.C. Cheung, G.L. Yu, S. Ruben, M. Murphy, R.J. Eisenberg, G.H. Cohen, P.G. Spear, and C.F. Ware. 1998. LIGHT, a new member of the TNF superfamily, and lymphotoxin alpha are ligands for herpesvirus entry mediator. Immunity. 8:21-30. doi:10.1016/S1074-7613(00)80455-0.
Montgomery, R.I., M.S. Warner, B.J. Lum, and P.G. Spear. 1996. Herpes simplex virus-1 entry into cells mediated by a novel member of the TNF/NGF receptor family. Cell. 87:427-436.
Morel, Y., A. Trunch, R.W. Sweet, D. Olive, and R.T. Costello. 2001. The TNF Superfamily Members LIGHT and CD154 (CD40 Ligand) Costimulate Induction of Dendritic Cell Maturation and Elicit Specific CTL Activity. The Journal of Immunology. 167:2479-2486. doi:10.4049/jimmunol.167.5.2479.
Murphy, K.M., C.A. Nelson, and J.R. Šedý. 2006. Balancing co-stimulation and inhibition with BTLA and HVEM. Nat Rev Immunol. 6:671-681. doi:10.1038/nri1917.
Murphy, T.L., and K.M. Murphy. 2010. Slow Down and Survive: Enigmatic Immunoregulation by BTLA and HVEM. Annu. Rev. Immunol. 28:389-411. doi:10.1146/annurev-immunol-030409-101202.
Nimmerjahn, F., P. Bruhns, K. Horiuchi, and J.V. Ravetch. 2005. FcgammaRIV: a novel FcR with distinct IgG subclass specificity. Immunity. 23:41-51. doi:10.1016/j.immuni.2005.05.010.
Petro, C., P.A. González, N. Cheshenko, and T. Jandl. 2015. Herpes simplex type 2 virus deleted in glycoprotein D protects against vaginal, skin and neural disease. eLife. doi:10.7554/eLife.06054.001.
Petro, C.D., B. Weinrick, N. Khajoueinejad, C. Burn, R. Sellers, W.R. Jacobs Jr, and B.C. Herold. 2016. HSV-2 ΔgD elicits FcγR-effector antibodies that protect against clinical isolates. JCI Insight. 1:1-15. doi:10.1172/jci.insight.88529.
Poole, E., C.A. King, J.H. Sinclair, and A. Alcami. 2006. The UL144 gene product of human cytomegalovirus activates NFKB via a TRAF6-dependent mechanism. EMBO J. 25:4390-4399. doi:10.1038/sj.emboj.7601287.
Roberts, C.M., J.R. Pfister, and S.J. Spear. 2003. Increasing Proportion of Herpes Simplex Virus Type 1 as a Cause of Genital Herpes Infection in College Students. Sexually Transmitted Diseases. 30:797-800. doi:10.1097/01.OLQ.0000092387.58746.C7.
Sarrias, M.R., J.C. Whitbeck, I. Rooney, C.F. Ware, R.J. Eisenberg, G.H. Cohen, and J.D. Lambris. 2000. The three HveA receptor ligands, gD, LT-alpha and LIGHT bind to distinct sites on HveA. Molecular Immunology. 37:665-673.
Scheu, S., J. Alferink, T. Potzel, W. Barchet, U. Kalinke, and K. Pfeffer. 2002. Targeted Disruption of LIGHT Causes Defects in Costimulatory T Cell Activation and Reveals Cooperation with Lymphotoxin ß in Mesenteric Lymph Node Genesis. Journal of Experimental Medicine. 195:1613-1624. doi:10.1084/jem.20020215.
Shi, G. 2002. Mouse T cells receive costimulatory signals from LIGHT, a TNF family member. Blood. 100:3279-3286. doi:10.1182/blood-2002-05-1404.
Shui, J.-W., and M. Kronenberg. 2013. HVEM: An unusual TNF receptor family member important for mucosal innate immune responses to microbes. Gut Microbes. 4:146-151. doi:10.4161/gmic.23443.
Spear, P.G. 2004. Herpes simplex virus: receptors and ligands for cell entry. Cell Microbiol. 6:401-410. doi:10.1111/j.1462-5822.2004.00389.x.
Stanberry, L.R., S.L. Spruance, A.L. Cunningham, D.I. Bernstein, A. Mindel, S. Sacks, S. Tyring, F.Y. Aoki, M. Slaoui, M. Denis, P. Vandepapeliere, G. Dubin, GlaxoSmithKline Herpes Vaccine Efficacy Study Group. 2002. Glycoprotein-D-adjuvant vaccine to prevent genital herpes. N Engl J Med. 347:1652-1661. doi:10.1056/NEJMoa011915.
Steinberg, M.W., T.C. Cheung, and C.F. Ware. 2011. The signaling networks of the herpesvirus entry mediator (TNFRSF14) in immune regulation. Immunological Reviews. 244:169-187. doi:10.1111/j.1600-065X.2011.01064.x.
Stiles, K.M., J.C. Whitbeck, H. Lou, G.H. Cohen, R.J. Eisenberg, and C. Krummenacher. 2010. Herpes Simplex Virus Glycoprotein D Interferes with Binding of Herpesvirus Entry Mediator to Its Ligands through Downregulation and Direct Competition. J. Virol. 84:11646-11660. doi:10.1128/JVI.01550-10.
Šedý, J., V. Bekiaris, and C.F. Ware. 2015. Tumor Necrosis Factor Superfamily in Innate Immunity and Inflammation. Cold Spring Harb Perspect Biol. 7: a016279-19. doi:10.1101/cshperspect.a016279.
Šedý, J.R., M. Gavrieli, K.G. Potter, M.A. Hurchla, R.C. Lindsley, K. Hildner, S. Scheu, K. Pfeffer, C.F. Ware, T.L. Murphy, and K.M. Murphy. 2004. B and T lymphocyte attenuator regulates T cell activation through interaction with herpesvirus entry mediator. Nat. Immunol. 6:90-98. doi:10.1038/ni1144.
Tamada, K., K. Shimozaki, A.I. Chapoval, Y. Zhai, J. Su, S.F. Chen, S.L. Hsieh, S. Nagata, J. Ni, and L. Chen. 2000. LIGHT, a TNF-Like Molecule, Costimulates T Cell Proliferation and Is Required for Dendritic Cell-Mediated Allogeneic T Cell Response. The Journal of Immunology. 164:4105-4110. doi:10.4049/jimmunol.164.8.4105.
Watanabe, N., M. Gavrieli, J.R. Šedý, J. Yang, F. Fallarino, S.K. Loftin, M.A. Hurchla, N. Zimmerman, J. Sim, X. Zang, T.L. Murphy, J.H. Russell, J.P. Allison, and K.M. Murphy. 2003. BTLA is a lymphocyte inhibitory receptor with similarities to CTLA-4 and PD-1. Nat. Immunol. 4:670-679. doi:10.1038/ni944.
Zanoni, I., R. Ostuni, G. Capuano, M. Collini, M. Caccia, A.E. Ronchi, M. Rocchetti, F. Mingozzi, M. Foti, G. Chirico, B. Costa, A. Zaza, P. Ricciardi-Castagnoli, and F. Granucci. 2009. CD14 regulates the dendritic cell life cycle after LPS exposure through NFAT activation. Nature. 460:264-268. doi:10.1038/nature08118.
Zhu, Y., S. Yao, M.M. Augustine, H. Xu, J. Wang, J. Sun, M. Broadwater, W. Ruff, L. Luo, G. Zhu, K. Tamada, and L. Chen. 2016. Neuron-specific SALM5 limits inflammation in the CNS via its interaction with HVEM. Science Advances. 2:e1500637-e1500637. doi:10.1126/sciadv.1500637.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A method of decreasing or blocking Fc-gamma receptor (FcγR)-mediated killing of self-antigen in a subject having an autoimmune disease comprising administering to the subject an amount of an HVEM antagonist, a soluble herpes simplex virus (HSV) glycoprotein D, an antibody binding HVEM, or a combination thereof, effective to decrease or block the FcγR-mediated killing in the subject.

31. A method of blocking Fc-gamma receptor (FcγR)-mediated killing of self-antigen in a subject having an autoimmune disease comprising administering to the subject an amount of an HVEM antagonist, a soluble herpes simplex virus (HSV) glycoprotein D, an antibody binding HVEM, or a combination thereof, effective to reduce the autoimmune disease in the subject.

32. The method of claim 30, wherein the soluble HSV glycoprotein D comprises HSV-1 glycoprotein D, HSV-2 glycoprotein D, or a combination thereof.

33. The method of claim 31, wherein the soluble HSV glycoprotein D comprises HSV-1 glycoprotein, HSV-2 glycoprotein D, or a combination thereof.

30. The method of claim 30, wherein the HVEM antagonist binds HVEM.

35. The method of claim 31, wherein the HVEM antagonist binds HVEM.

36. A kit for decreasing or blocking Fc-gamma receptor (FcγR)-mediated killing of self-antigen in a subject having an autoimmune disease comprising an HVEM antagonist, a soluble HSV glycoprotein D, an antibody binding HVEM, or a combination thereof.

37. The kit according to claim 36, wherein the soluble glycoprotein D comprises HSV-2 glycoprotein D, HSV-2 glycoprotein D, or a combination thereof.

38. The kit according to claim 36, wherein the HVEM antagonist binds HVEM.
